(19) United States
(12) Patent Application Publication
(10) Pub. No.: US 2023/0210829 A1
(43) Pub. Date: Jul. 6, 2023

(54) REJUVENATION OF AGED TISSUES BY INHIBITION OF THE PGE2 DEGRADING ENZYME, 15-PGDH

(71) Applicant: The Board of Trustees of the Leland Stanford Junior University, Stanford, CA (US)

(72) Inventors: Helen M. Blau, Stanford, CA (US); Adelaida Rosa Palla, Stanford, CA (US); Yu Xin Wang, Stanford, CA (US)

(73) Assignee: The Board of Trustees of the Leland Stanford Junior University, Stanford, CA (US)

(21) Appl. No.: 18/008,750

(22) PCT Filed: Jun. 11, 2021
(86) PCT No.: PCT/US2021/037068

§ 371 Date: Dec. 7, 2022

Related U.S. Application Data
(60) Provisional application No. 63/037,852, filed on Jun. 11, 2020.

(51) Int. Cl. A61K 31/4365; A61P 43/00

(52) U.S. Cl. CPC A61K 31/4365; A61P 43/00

(57) ABSTRACT

The present disclosure provides compositions and methods based on the use of 15-PGDH as a therapeutic target in rejuvenation of aging non-skeletal muscle tissues and/or organs. The 15-PGDH inhibitor SW033291 administered intraperitoneally for 4 weeks resulted in restoration of follicular structure and re-establishment of the marginal zone in spleens of 25 month old mice. Treatment of 25 month old mice with SW033291 also reduced the levels of IL10, IL6, BTC, GM-CSF, IL 13 back to levels similar 0 to 4 month old mice.

### CROSS-REFERENCE

This application claims priority to U.S. Provisional Patent Application No. 63/037,852, filed Jun. 11, 2020, the disclosure of which is herein incorporated by reference in its entirety.

### STATEMENT AS TO RIGHTS TO INVENTIONS MADE UNDER FEDERALLY SPONSORED RESEARCH AND DEVELOPMENT

This invention was made with Government support under contracts AG020961 and AG069858 awarded by the National Institutes of Health. The Government has certain rights in the invention.

### BACKGROUND

As we age, quality of life is reduced and mortality is increased. Age-related diseases are a group of diseases that occur more frequently in people as they age, and which can also be caused or accelerated by genetic or other factors, which directly correlate to decreased longevity. Aging and age-related diseases and conditions can have numerous deleterious effects on tissues, leading to an overall loss of function or health of the tissues.

Prostaglandin E2 (PGE2), also known as dinoprostone, has been employed in various clinical settings including to induce labor in women and to augment hematopoietic stem cell transplantation. PGE2 can be used as an anticoagulant and antithrombotic agent. The role of PGE2 as a lipid mediator that can resolve inflammation is also well known. Nonsteroidal anti-inflammatory drugs (NSAIDs), inhibitors of COX-1 and/or COX-2, suppress inflammation by inhibiting prostanoids, mainly via PGE2 biosynthesis. Prostaglandin D2 (PGD2) is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. PGE2 and PGD2 are both synthesized from arachidonic acid by cyclooxygenases (COX) and by prostaglandin E synthase enzymes or prostaglandin D synthase enzymes, respectively. Levels of PGE2 and PGD2 are physiologically regulated by the enzyme 15-hydroxyprostaglandin dehydrogenase (15-PGDH), which catalyzes the conversion of the 15-OH group of both PGE2 and PGD2 to a 15-keto group.

### BRIEF SUMMARY

There remains a need in the art for effective treatments for preventing or reversing loss of function in tissues and/or organs, e.g., non-skeletal muscle tissues, in subjects with age-related diseases and disorders and/or in aged subjects. The present disclosure satisfies this need and provides other advantages as well.

The present disclosure provides compositions and methods for improving the health, function, and/or performance of non-skeletal muscle tissues and/or organs in subjects with age-related conditions or diseases and/or in aged subjects, in particular by inhibiting 15-PGDH in the subjects.

In one aspect, a method of rejuvenating a function of an aged non-skeletal muscle tissue or aged non-skeletal muscle organ in an individual is provided, the method comprising: administering to the individual or to the aged non-skeletal muscle tissue or aged non-skeletal muscle organ a 15-hydroxyprostaglandin dehydrogenase (15-PGDH) inhibitor in an amount effective to inhibit 15-PGDH activity and/or reduce 15-PGDH levels in the individual, thereby rejuvenating the function of the aged non-skeletal muscle tissue or the aged non-skeletal muscle organ in the individual. In some cases, after the administering, the function is rejuvenated relative to a function of the aged non-skeletal muscle tissue or aged non-skeletal muscle organ prior to administration of the 15-PGDH inhibitor. In some cases, after the administering, the function is rejuvenated by at least about 10% relative to a function of the aged non-skeletal muscle tissue or aged non-skeletal muscle organ prior to administering the 15-PGDH inhibitor. In some cases, after the administering, the function of the aged non-skeletal muscle tissue or the aged non-skeletal muscle organ is rejuvenated to a level substantially similar to a level of a function of a young non-skeletal muscle tissue or a young non-skeletal muscle organ. In some cases, after the administering, the function of the aged non-skeletal muscle tissue or the aged non-skeletal muscle organ is rejuvenated to a level within at least about 50% of a level of a function of a young non-skeletal muscle tissue or a young non-skeletal muscle organ. In some cases, after the administering, a level of prostaglandin E2(PGE2) in the aged non-skeletal muscle tissue or aged non-skeletal muscle organ is increased relative to a level of PGE2 in the aged non-skeletal muscle tissue or aged non-skeletal muscle organ prior to the administering. In some cases, after the administering, a level of PGE2 in the aged non-skeletal muscle tissue or aged non-skeletal muscle organ is increased by at least about 10% relative to a level of PGE2 in the aged non-skeletal muscle tissue or aged non-skeletal muscle organ prior to the administering. In some cases, after the administering, a level of PGE2 in the aged non-skeletal muscle tissue or aged non-skeletal muscle organ is increased to a level substantially similar to a level of PGE2 present in a young non-skeletal muscle tissue or a young non-skeletal muscle organ. In some cases, after the administering, a level of PGE2 in the aged non-skeletal muscle tissue or aged non-skeletal muscle organ is increased to a level within at least about 50% of a level of PGE2 present in a young non-skeletal muscle tissue or a young non-skeletal muscle organ. In some cases, the administering increases systemic levels of PGE2 in the individual. In some cases, the administering results in a rejuvenation of serum cytokines to levels substantially similar to serum cytokine levels found in a young individual. In some cases, the serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-a), interleukin-1 beta (IL1b), interleukin-22 (IL22), and any combination thereof. In some cases, the aged non-skeletal muscle tissue or the aged non-skeletal muscle organ is selected from the group consisting of: epidermal tissue, epithelial tissue, vascular tissue, cardiac muscle, brain, bone, cartilage, sensory organs (e.g., organs involved in sight, hearing, taste, smell, or touch), kidney, thyroid, lung, smooth muscle, brown fat, spleen, liver, heart, small intestine, colon, skin, ovaries and other reproductive tissues, hair, dental tissue, blood, cochlea, and any combination thereof. In some cases, the aged non-skeletal muscle tissue or the aged non-skeletal muscle organ is spleen. In some cases, after the administering, the spleen exhibits improved clearance of pathogens, microorganisms, cellular debris, and/or aged erythrocytes from the blood, improved or enhanced maturation of lymphoid cell types, increased antibody generation, or any combination thereof. In some cases, the administering results in: increased adaptive and/or innate immune response in the individual relative to prior to the administering, decreased severity of infection in the individual relative to prior to the administering, decreased production of auto-antibodies relative to prior to the administering, treatment of or improvement of symptoms associated with type II diabetes, treatment of or improvement of symptoms associated with rheumatoid arthritis, or any combination thereof. In some cases, the aged non-skeletal muscle tissue or the aged non-skeletal muscle organ is skin. In some cases, after the administering, the skin exhibits enhanced skin condition, exhibits increased barrier function, supports increased hair growth, counters baldness, exhibits increased stimulation of hair follicle stem cells, exhibits increased elasticity of skin, treatment or improvement of symptoms or effects associated with alopecia, treatment or improvement of symptoms or effects associated with pattern baldness, or any combination thereof. In some cases, the aged non-skeletal muscle tissue or the aged non-skeletal muscle organ is brain. In some cases, after the administering, the brain exhibits increased brain size, exhibits increased grey matter, exhibits increased amount of neuronal cells, exhibits increased neuronal volume, exhibits improved cognitive performance, exhibits improved memory performance, exhibits increased level of neurotransmitters such as dopamine, serotonin and other brain-derived neurotrophic factors, exhibits increased level of hormones, exhibits reduced risk of stroke, white matter lesions, or dementia, exhibits reduced risk of Alzheimer's or Parkinson's disease, or any combination thereof. In some cases, the aged non-skeletal muscle tissue or the aged non-skeletal muscle organ is bone. In some cases, after the administering, the bone exhibits improved mechanical support and movement, exhibits improved angiogenesis, exhibits improved storage of mineral or fat, exhibits improved stabilization of pH or calcium, exhibits improved hormone secretion, exhibits improved lubrication, exhibits decreased fibrosis, or any combination thereof. In some cases, the aged non-skeletal muscle tissue or the aged non-skeletal muscle organ is kidney. In some cases, after the administering, the kidney is protected from ischemic renal injury, exhibits increased vasodilation, exhibits increased renal blood flow, exhibits reduced biomarkers of renal injury, exhibits induction of PGE2 levels, exhibits induction of PGE2 receptors, exhibits improved formation of urine, exhibits improved filtration, exhibits improved reabsorption, exhibits improved secretion, exhibits improved excretion, exhibits improved hormone secretion, exhibits improved blood pressure regulation, exhibits improved acid-base balance, exhibits improved regulation of osmolality, exhibits decreased levels of kidney disease, or any combination thereof. In some cases, the aged non-skeletal muscle tissue or the aged non-skeletal muscle organ is thyroid. In some cases, after the administering, the thyroid exhibits improved regulation, production, and/or secretion of hormones and/or exhibits decreased levels of thyroid disease. In some cases, the aged non-skeletal muscle tissue or the aged non-skeletal muscle organ is lung. In some cases, after the administering, the lung exhibits decreased levels of lung disease, exhibits decreased levels of fibrosis, exhibits increased lung capacity, or any combination thereof. In some cases, the aged non-skeletal muscle tissue or the aged non-skeletal muscle organ is cardiac muscle and/or heart. In some cases, after the administering, the cardiac muscle and/or heart exhibits increased or enhanced cardiac muscle tissue functions, improved pumping of oxygenated blood to other body parts, improved pumping of hormones and other vital substances to different parts of the body, improvement in receiving deoxygenated blood and carrying metabolic waste products from the body and pumping it to the lungs for oxygenation, improved maintenance of blood pressure, exhibits decreased fibrosis, or any combination thereof. In some cases, the aged non-skeletal muscle tissue or the aged non-skeletal muscle organ is smooth muscle. In some cases, after the administering, the smooth muscle exhibits decreased levels of smooth muscle disease and/or decreased levels of fibrosis, exhibits increased angiogenesis or vasculogenesis, and/or is more sensitive to temperature changes and/or adrenalin level changes. In some cases, the aged non-skeletal muscle tissue or the aged non-skeletal muscle organ is liver. In some cases, after the administering, the liver exhibits improved maintenance of whole-body homeostasis through regulation of metabolism, xenobiotic, and endobiotic clearance and molecular biosynthesis, exhibits improved formation and excretion of bile, exhibits improved regulation of carbohydrate homeostasis, lipid synthesis and secretion of plasma lipid proteins, exhibits improved control of cholesterol metabolism, exhibits improved formation of urea, serum albumin, clotting factors, enzymes, and other proteins, exhibits decreased fibrosis, exhibits decreased fatty acid storage, exhibits decreased liver adipose content, or any combination thereof. In some cases, the aged non-skeletal muscle tissue or the aged non-skeletal muscle organ is small intestine. In some cases, after the administering, the small intestine exhibits increased production of lactase, exhibits reduced growth of certain bacteria, improved digestion of dairy products, improved absorption of nutrients, or any combination thereof. In some cases, the aged non-skeletal muscle tissue or the aged non-skeletal muscle organ is colon. In some cases, after the administering, the colon exhibits increased or enhanced peristalsis and/or recovery from ulcerative colitis including diarrhea or gastrointestinal bleeding. In some cases, the aged non-skeletal muscle tissue or the aged non-skeletal muscle organ is ovaries or other reproductive tissues/organs. In some cases, after the administering, the ovaries or other reproductive tissues/organs exhibit reduced or halted ovary decline and/or exhibits a reduction in pregnancy failure and/or number of chromosomally aberrant conceptions. In some cases, the aged non-skeletal muscle tissue or the aged non-skeletal muscle organ is hair. In some cases, a property of the aged hair is rejuvenated, the property selected from the group consisting of: pigmentation, diameter, curvature, stretching, bending, torsional rigidity, lipid composition, and any combination thereof. In some cases, the aged non-skeletal muscle tissue or the aged non-skeletal muscle organ is dental tissue. In some cases, after the administering, the dental tissue exhibits an increased ratio of dentin to dental pulp and/or a reduced level or reversal of the conversion of dental pulp to dentin. In some cases, the aged non-skeletal muscle tissue or the aged non-skeletal muscle organ is blood. In some cases, after the administering, the blood exhibits improved supply of oxygen to tissues, exhibits improved supply of nutrients to tissues, exhibits improved removal of waste, exhibits improved immune response, exhibits improved circulation of white blood cells, exhibits improved detection of foreign material by antibodies, exhibits improved coagulation, exhibits improved transport of hormones, exhibits improved regulation of core body temperature, exhibits decreased levels of blood diseases, or any combination thereof. In some cases, the aged non-skeletal muscle tissue or the aged non-skeletal muscle organ is a sensory organ or the cochlea. In some cases, after the administering, the sensory organ exhibits enhanced or improved sensory function (e.g., sight, smell, taste, hearing) and/or reduction or treatment of dry eye disease, lacrimal gland inflammation, or macular degeneration. In some cases, the aged non-skeletal muscle tissue or the aged non-skeletal muscle organ is epithelial tissue. In some cases, after the administering, the epithelial tissue exhibits improved secretion, exhibits improved selective absorption, exhibits improved protection of underlying tissues (e.g., from radiation, desiccation, toxins, invasion by pathogens, physical trauma), exhibits improved transcellular transport, exhibits improved sensing, or any combination thereof. In some cases, the aged non-skeletal muscle tissue or the aged non-skeletal muscle organ is vascular tissue. In some cases, after the administering, the vascular tissue exhibits improved vasodilation, improved angiogenesis, improved access of nutrients to tissue, improved blood transport, or any combination thereof. In some cases, the individual has one or more biomarkers of aging. In some cases, the one or more biomarkers of aging is selected from the group consisting of: an increase in 15-PGDH levels relative to a young individual, a decrease in PGE2 levels relative to a young individual, an increase in a PGE2 metabolite relative to a young individual, an increase or a greater accumulation of senescent cells relative to a young individual, an increase in expression of one or more atrogenes relative to a young individual, a decrease in mitochondria biogenesis and/or function relative to a young individual, an increase in transforming growth factor pathway signaling relative to a young individual, and any combination thereof. In some cases, the aged non-skeletal muscle tissue or the aged non-skeletal muscle organ has an increased accumulation of senescent cells relative to a young non-skeletal muscle tissue or a young non-skeletal muscle organ. In some cases, the senescent cells express one or more senescent markers. In some cases, the senescent cells have an increased level of one or more senescent markers relative to non-senescent cells. In some cases, the one or more senescent markers is selected from the group consisting of: p15Ink4b, p16Ink4a, p19Arf, p21, Mmp13, Il1a, Il1b, and Il6. In some cases, the senescent cells are macrophages. In some cases, the 15-PGDH inhibitor is selected from the group consisting of: a small molecule compound, a blocking antibody, a nanobody, and a peptide. In some cases, the 15-PGDH inhibitor is SW033291. In some cases, the 15-PGDH inhibitor is selected from the group consisting of: an antisense oligonucleotide, microRNA, siRNA, and shRNA. In some cases, the individual is a human. In some cases, the individual is at least 30 years of age. In some cases, the 15-PGDH inhibitor reduces or blocks 15-PGDH expression. In some cases, the 15-PGDH inhibitor reduces or blocks enzymatic activity of 15-PGDH. In some cases, the administering results in decreased levels of a PGE2 metabolite in the aged non-skeletal muscle tissue or aged non-skeletal muscle organ relative to the aged non-skeletal muscle tissue or the aged non-skeletal muscle muscle prior to the administering of the 15-PGDH inhibitor and/or to a level that is substantially similar to a level present in young non-skeletal muscle tissue or a young non-skeletal muscle organ. In some cases, the PGE2 metabolite is selected from the group consisting of: 15-keto PGE2 and 13,14-dihydro-15-keto PGE2. In some cases, the administering comprises systemic administration. In some cases, the systemic administration is oral administration or intraperitoneal administration. In some cases, the administering comprises local administration. In some cases, the administering comprises single-dose administration. In some cases, the administering comprises administering the 15-PGDH inhibitor periodically.

In one aspect, a method of rejuvenating an aged non-skeletal muscle tissue in a subject is provided, the method comprising: administering to the subject an amount of a 15-PGDH inhibitor effective to inhibit 15-PGDH activity and/or reduce 15-PGDH levels in the subject, thereby rejuvenating the aged non-skeletal muscle tissue. In some cases, the administering increases a level of PGE2 in the aged non-skeletal muscle tissue of the subject. In some cases, a level of PGE2 in the aged non-skeletal muscle tissue is increased relative to the aged non-skeletal muscle tissue prior to the administering of the 15-PGDH inhibitor. In some cases, a level of PGE2 in the aged non-skeletal muscle tissue is increased by at least 10% relative to the aged non-skeletal muscle tissue prior to the administering of the 15-PGDH inhibitor. In some cases, a of PGE2 in the aged non-skeletal muscle tissue is increased to a level substantially similar to a level present in young non-skeletal muscle tissue. In some cases, a level of PGE2 in the aged non-skeletal muscle tissue is increased to a level within 50% of a level present in young non-skeletal muscle tissue. In some cases, the aged non-skeletal muscle tissue is selected from the group consisting of: epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof. In some cases, the subject has one or more biomarkers of aging. In some cases, the one or more biomarkers of aging is selected from the group consisting of: an increase in 15-PGDH levels relative to young non-skeletal muscle tissue, a decrease in PGE2 levels relative to young non-skeletal muscle tissue, an increase in a PGE2 metabolite relative to young non-skeletal muscle tissue, an increase or a greater accumulation of senescent cells relative to young non-skeletal muscle tissue, an increase in expression of one or more atrogenes relative to young non-skeletal muscle tissue, a decrease in mitochondria biogenesis and/or function relative to young non-skeletal muscle tissue, and an increase in transforming growth factor pathway signaling relative to young non-skeletal muscle tissue. In some cases, the aged non-skeletal muscle tissue has an increased accumulation of senescent cells relative to young non-skeletal muscle tissue. In some cases, the senescent cells express one or more senescent markers. In some cases, the senescent cells have an increased level of one or more senescent markers relative to non-senescent cells. In some cases, the one or more senescent markers is selected from the group consisting of: p15Ink4b, p16Ink4a, p19Arf, p21, Mmp13, Il1a, Il1b, and Il6. In some cases, the senescent cells are macrophages. In some cases, the method further comprises administering a senolytic agent to the aged non-skeletal muscle tissue. In some cases, the senolytic agent is selected from the group consisting of: a Bcl2 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof. In some cases, the 15-PGDH inhibitor is selected from the group consisting of: a small molecule compound, a blocking antibody, a nanobody, and a peptide. In some cases, the 15-PGDH inhibitor is SW033291. In some cases, the 15-PGDH inhibitor is selected from the group consisting of: an antisense oligonucleotide, microRNA, siRNA, and shRNA. In some cases, the subject is a human. In some cases, the subject is at least 30 years of age. In some cases, the 15-PGDH inhibitor reduces or blocks 15-PGDH expression. In some cases, the 15-PGDH inhibitor reduces or blocks enzymatic activity of 15-PGDH. In some cases, a function of the aged non-skeletal muscle is enhanced relative to the function of the aged non-skeletal muscle prior to the administering of the 15-PGDH inhibitor. In some cases, a function of the aged non-skeletal muscle tissue is enhanced by at least 10% relative to the function of the aged non-skeletal muscle prior to the administering of the 15-PGDH inhibitor. In some cases, a function of the aged non-skeletal muscle tissue is enhanced to a level that is substantially similar to a level present in young non-skeletal muscle tissue. In some cases, a function of the aged non-skeletal muscle tissue is enhanced to a level that is within 50% of a level present in young non-skeletal muscle tissue. In some cases, the function comprises increased protein synthesis, increased cell proliferation, increased cell survival, decreased protein degradation, or any combination thereof. In some cases, the method results in decreased levels of a PGE2 metabolite in the aged non-skeletal muscle tissue relative to the aged non-skeletal muscle tissue prior to the administering of the 15-PGDH inhibitor and/or to a level that is substantially similar to a level present in young non-skeletal muscle. In some cases, the PGE2 metabolite is selected from the group consisting of: 15-keto PGE2 and 13,14-dihydro-15-keto PGE2.

In another aspect, a method of enhancing a function of a non-skeletal muscle tissue in a subject is provided, the method comprising: administering to the subject a 15-PGDH inhibitor in an amount effective to inhibit 15-PGDH activity and/or reduce 15-PGDH levels in the non-skeletal muscle tissue, thereby enhancing a function of the non-skeletal muscle tissue in the subject. In some cases, the function is enhanced relative to the non-skeletal muscle tissue prior to the administering of the 15-PGDH inhibitor. In some cases, the function is an increase in protein synthesis, an increase in cell proliferation, an increase in cell survival, a decrease in protein degradation, or any combination thereof. In some cases, the subject is less than 30 years of age. In some cases, the subject is greater than 30 years of age.

In another aspect, the present disclosure provides a method for increasing the function of a non-skeletal muscle tissue in a subject with an age-related disorder, the method comprising administering to the subject a therapeutically effective amount of a 15-hydroxyprostaglandin dehydrogenase (15-PGDH) inhibitor, wherein the administration of the 15-PGDH inhibitor increases or restores the level of PGE2 and/or PGD2 in the non-skeletal muscle tissue in the subject.

In some embodiments of the method, the age-related disorder is selected from the group consisting of cardiovascular disease, chronic respiratory disease, nutritional disease, kidney disease, gastrointestinal or digestive disease, neurological disorder, sensory disorder, hearing disorder, skin or subcutaneous disease, cerebrovascular disease, osteoporosis, osteoarthritis, premature aging disease, and combinations thereof. In some embodiments, the cardiovascular disease is atrial fibrillation, stroke, ischemic heart disease, cardiomyopathy, endocarditis, intracerebral hemorrhage, hypertension, or a combination thereof. In some embodiments, the chronic respiratory disease is chronic obstructive pulmonary disease, asbestosis, silicosis, or a combination thereof. In some embodiments, the nutritional disease is trachoma, diarrheal disease, encephalitis, or a combination thereof. In some embodiments, the kidney disease is a chronic kidney disease. In some embodiments, the gastrointestinal or digestive disease is NASH, pancreatitis, ulcer, intestinal obstruction, or a combination thereof. In some embodiments, the neurological disorder is Alzheimer's disease, dementia, Parkinson's disease, or a combination thereof. In some embodiments, the sensory disorder is hearing loss, vision loss, loss of sense of smell or sense of taste, macular degeneration, retinosa pigmentosa, glaucoma, or a combination thereof. In some embodiments, the skin or subcutaneous disease is cellulitis, ulcer, fungal skin disease, pyoderma, or a combination thereof. In some embodiments, the premature aging disease is Osteogenesis imperfecta, Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, Rothmund-Thomson syndrome, an immunodeficiency leading to a premature aging syndrome such as Ataxia telangiectasia, or an infectious disease leading to premature aging such as HIV.

In some embodiments of the method, the subject is a human. In some embodiments, the method further comprises a step in which the human is selected for treatment with the 15-PGDH inhibitor based on a diagnosis of the age-related disorder. In some embodiments, the non-skeletal muscle tissue is selected from the group consisting of epidermal, epithelial, vascular, cardiac muscle, brain, bone, cartilage, sensory organs, kidney, thyroid, lung, smooth muscle, brown fat, spleen, liver, heart, brain, small intestine, colon, skin, ovaries and other reproductive tissues, hair, dental tissues, cochlea, oligodendrocytes, and combinations thereof.

In some embodiments of the method, the 15-PGDH inhibitor inactivates 15-PGDH or blocks 15-PGDH activity. In some embodiments, the 15-PGDH inhibitor reduces or blocks the enzymatic activity of 15-PGDH. In some embodiments, the 15-PGDH inhibitor is a small molecule compound, blocking antibody, nanobody, or peptide. In some embodiments, the small molecule compound is SW033291. In some embodiments, the 15-PGDH inhibitor reduces or blocks 15-PGDH expression. In some embodiments, the 15-PGDH inhibitor is an antisense oligonucleotide, microRNA, siRNA, or shRNA.

In some embodiments of the method, the administration of the 15-PGDH inhibitor increases or restores the level of PGE2 in the non-skeletal muscle tissue in the subject. In some embodiments, the therapeutically effective amount of the 15-PGDH inhibitor decreases PGE2 and/or PGD2 metabolite levels in the non-skeletal muscle tissue of the subject. In some embodiments, the PGE2 metabolite is 15-keto-PGE2 or 13,14-dihydro-15-keto-PGE2 (PGEM). In some embodiments, the PGD2 metabolite is 15-keto-PGD2 or 13,14-dihydro-15-keto-PGD2. In some embodiments, the therapeutically effective amount of the 15-PGDH inhibitor increases protein synthesis, increases cell proliferation, increases cell survival, lengthens telomeres, and/or decreases protein degradation, in the non-skeletal muscle tissue of the subject. In some embodiments, administering the 15-PGDH inhibitor comprises systemic administration. In some embodiments, administering the 15-PGDH inhibitor comprises local administration. In some embodiments, the non-skeletal muscle tissue has an increased accumulation of senescent cells relative to young non-skeletal muscle tissue. In some embodiments, the method further comprises administering a senolytic agent to the subject. In some embodiments, the senolytic agent is selected from the group consisting of a Bcl2 inhibitor, a pan-tyrosine kinase inhibitor, a flavonoid, a peptide that interferes with the FOXO4-p53 interaction, a selective targeting system of senescent cells using galactooligosaccharide-coated nanoparticles, an HSP90 inhibitor, and combinations thereof.

Other objects, features, and advantages of the present disclosure will be apparent to one of skill in the art from the following detailed description and figures.

### BRIEF DESCRIPTION OF THE DRAWINGS

FIGS. 1A and 1B. PGE2 degrading enzyme 15-PGDH is increased in aged tissues. (FIG. 1A) PGE2 and PGD2 catabolism scheme. (FIG. 1B) 15-PGDH specific enzymatic activity assayed in tissues of young (2 months) and aged (25 months) mice. Activity is expressed as percent change relative to young. *P<0.05, **P<0.001, ***P<0.0005. Multiple t-tests (FIG. 1B). Means±s.e.m. Abbreviations: Spl. Spleen: Mus. Muscle.

FIG. 2. 15-PGDH specific activity assay of young and aged tissues. Kinetic measurement of 15-PGDH specific activity in lysates prepared from young (gray) and aged (black) tissues.

FIG. 3. Inhibition of 15-PGDH rejuvenates splenic morphology. Hematoxylin and Eosin (H&E) staining of spleen in young vehicle treated (4 months), aged vehicle treated (25 months) and aged SW033291 treated (25 months) intraperitoneally for 4 weeks.

FIGS. 4A-4H. Inhibition of 15-PGDH rejuvenates systemic cytokine levels. Serum levels of (FIG. 4A) interleukin-10 (IL10), (FIG. 4B) interleukin-6 (IL6), (FIG. 4C) betacellulin (BTC), (FIG. 4D) granulocyte-macrophage colony-stimulating factor (GM-CSF), (FIG. 4E) interleukin-13 (IL13), (FIG. 4F) tumor necrosis factor-alpha (TNFA), (FIG. 4G) interleukin-1 beta (IL1B), and (FIG. 4H) interleukin-22 (IL22) measured in young vehicle (4 months), young SW033291 (4 months), aged vehicle treated (25 months), and aged SW033291 treated (25 months) mice by Luminex bead arrays. MFI—Mean fluorescence intensity.

### DETAILED DESCRIPTION

### 1. Introduction

The present disclosure is based, in part, on the discovery that the PGE2 degrading enzyme, 15-PGDH, or its transcript, is elevated in a range of aging tissues and/or organs, in particular, non-skeletal muscle tissues and/or organs. As such, 15-PGDH proteins or transcripts can be used as a biomarker for aging in non-skeletal muscle tissues and/or organs, e.g., in subjects with an age-related disorder or disease and/or in aged subjects. The present disclosure is also based on the discovery that inhibition of 15-PGDH can rejuvenate functions of aged non-skeletal muscle tissues and organs (e.g., such that the aged non-skeletal muscle tissues and organs function more similarly to young non-skeletal muscle tissues and organs). 15-PGDH can be inhibited in order to reverse or slow aging and aging-related processes in non-skeletal muscle tissues and/or organs, thereby ameliorating their function. Without being bound by the following theory, it is believed that elevated 15-PGDH levels in non-skeletal muscle tissues and/or organs in subjects with age-related conditions or diseases and/or in aged subjects, e.g., in the colon, brain, skin, spleen, or liver, leads to PGE2 and/or PGD2 degradation in these tissues and/or organs and thus to lower levels of PGE2 and/or PGD2 and of PGE2 and/or PGD2 signaling, which has deleterious effects on tissue function that are manifested in aging. The present disclosure therefore provides compositions and methods based on the use of 15-PGDH activity as a therapeutic target in non-skeletal muscle tissues and/or organs in subjects with age-related diseases or conditions and/or in aged subjects. Inhibiting 15-PGDH in these tissues and/or organs may restore or increase PGE2 and/or PGD2 levels in the tissues and/or organs and may ameliorate their function, health, and/or physiological activity. Reducing 15-PGDH can thus lead to improved quality of life and outcomes for age-related diseases or disorders.

A non-limiting list of aged non-skeletal muscle tissues and/or organs that can be treated using the present methods and compositions include, for example, epidermal tissue, vascular tissue, cardiac muscle, brain, bone, cartilage, smooth muscle, brown fat, spleen, liver, and the like. 15-PGDH elevation may occur in diseases of aged tissues and/or organs including cardiovascular diseases (e.g., atrial fibrillation, stroke, ischemic heart diseases, cardiomyopathies, endocarditis, intracerebral hemorrhage), chronic respiratory diseases (e.g., chronic obstructive pulmonary disease, asbestosis, silicosis), nutritional diseases (trachoma, diarrheal diseases, encephalitis), kidney diseases (e.g., chronic kidney diseases), gastrointestinal and digestive diseases (e.g., NASH, pancreatitis, ulcer, intestinal obstruction), neurological disorders (e.g., Alzheimer's, dementia, Parkinson's), sensory disorders (e.g., hearing loss, macular degeneration, glaucoma), skin and subcutaneous diseases (e.g., cellulitis, ulcer, fungal skin diseases, pyoderma), osteoporosis, osteoarthritis, rheumatoid arthritis and the like. In addition, genetic disorders of these tissues that lead to premature aging syndromes, such as Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, and Rothmund-Thomson syndrome, as well as immunodeficiencies of these tissues that lead to premature aging syndromes, such as Ataxia telangiectasia, and infectious diseases of these tissues that lead to premature aging syndromes, such as human immunodeficiency virus (HIV), can also benefit from 15-PGDH inhibition. 15-PGDH elevation in tissues and/or organs may also occur naturally during the aging process, and the methods and compositions provided herein contemplate treating a subject having aged tissues and/or organs with a 15-PGDH inhibitor, or by treating the aged tissue and/or organ itself with a 15-PGDH inhibitor, thereby rejuvenating the aged tissue and/or organ.

Treating non-skeletal muscle tissues and/or organs with inhibitors of 15-PGDH may provide numerous advantages, such as that the treatment can be localized to specific cell types that express elevated levels of the enzyme (e.g., diseased or aged non-skeletal muscle tissues and/or organs), that it provides the ability to restore endogenous levels of PGE2 and/or PGD2 to achieve physiological “youthful” levels of PGE2 and/or PGD2, that it can target non-skeletal muscle tissues and/or organs with high senescent cell infiltration (e.g., colon, skin, spleen), which is thought to have detrimental effects in aging and aging-associated conditions, and that it provides the possibility of targeting 15-PGDH with molecules with relatively long half-lives or by using gene therapy, in order to provide sustained, systemic PGE2 and/or PGD2 benefits.

### 2. General

Practicing the methods disclosed herein utilizes routine techniques in the field of molecular biology. Basic texts disclosing the general methods of use described herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel el al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy. e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.0 X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“Age-related condition” or “age-related disease” refers to any disease, condition, or disorder that shows or potentially shows any signs or features associated with increasing age or passage of time in non-skeletal muscle tissues and/or organs, including, e.g., loss or decrease of tissue and/or organ function, loss or decrease of tissue and/or organ health, loss or decrease of one or more physiological activities of the tissue and/or organ, decreased protein synthesis in cells of the tissue and/or organ, increased protein degradation in cells of the tissue and/or organ, decreased survival or viability of the tissue and/or organ, decreased proliferation of cells within the tissue and/or organ, shortened telomeres in cells of the tissue and/or organ, mitochondrial dysfunction in cells of the tissue and/or organ, increased presence of senescent cells in the tissue and/or organ, increased levels of 15-PGDH levels and/or activity in the tissue and/or organ, decreased levels of PGE2 and/or PGD2 in the tissue and/or organ, etc. The condition or disease can be a result of natural aging processes due to the passage of time, of other factors such as lifestyle factors or disease, e.g., infectious disease, or of genetic conditions that cause premature aging.

A “non-skeletal muscle tissue” or “non-skeletal muscle organ” as used herein can refer to any tissue or organ in the body other than skeletal muscle (e.g., other than musculi pectoralis complex, latissimus dorsi, teres major and subscapularis, brachioradialis, biceps, brachialis, pronator quadratus, pronator teres, flexor carpi radialis, flexor carpi ulnaris, flexor digitorum superficialis, flexor digitorum profundus, flexor pollicis brevis, opponens pollicis, adductor pollicis, flexor pollicis brevis, iliopsoas, psoas, rectus abdominis, rectus femoris, gluteus maximus, gluteus medius, medial hamstrings, gastrocnemius, lateral hamstring, quadriceps mechanism, adductor longus, adductor brevis, adductor magnus, gastrocnemius medial, gastrocnemius lateral, soleus, tibialis posterior, tibialis anterior, flexor digitorum longus, flexor digitorum brevis, flexor hallucis longus, extensor hallucis longus, ocular muscles, pharyngeal muscles, sphincter muscles, hand muscles, arm muscles, foot muscles, leg muscles, chest muscles, stomach muscles, back muscles, buttock muscles, shoulder muscles, head and neck muscles), and can encompass tissues (e.g., groups of cells that have similar structure and function together as a unit) and organs (e.g., two or more tissues that function in a particular manner), as well as particular cell types within an organ or tissue. For example, a “non-skeletal muscle tissue” or “non-skeletal muscle organ” can include any of the following: epithelial tissue, nerve tissue, connective tissue, smooth muscle, cardiac muscle, epidermal tissue, vascular tissue, heart, kidney, brain, bone, cartilage, brown fat, spleen, liver, colon, sensory organs, thyroid, lung, blood, small intestine, dental tissue, ovaries or other reproductive tissue or organs, hair, cochlea, oligodendrocytes, and combinations thereof.

The terms “aged tissue” and “aged organ” as used herein refer to any tissue or organ (e.g., non-skeletal muscle tissue or non-skeletal muscle organ) that exhibits one or more characteristics of a tissue or organ affected by an age-related condition, an age-related disease or disorder, and/or by the natural aging processes due to the passage of time. In some cases, the aged tissue or aged organ has increased levels and/or activity of 15-PGDH. In some cases, the aged tissue or aged organ has decreased levels of PGE2 and/or PGD2.

The terms “prostaglandin E2”, “PGE2”, and “dinoprostone” are used interchangeably herein and refer to prostaglandin that can be synthesized from arachidonic acid via cyclooxygenase (COX) enzymes and terminal prostaglandin E synthases (PGES). PGE2 plays a role in a number of biological functions including vasodilation, inflammation, and modulation of sleep/wake cycles. Structural and functional information about PGE2 can be found, e.g., in the entry for “Dinoprostone” of PubChem: pubchem.ncbi.nlm.nih.gov/compound/Dinoprostone, the contents of which are herein incorporated by reference in their entirety.

The terms “prostaglandin D2” or “PGD2” are used interchangeably herein and refer to prostaglandin that can be synthesized from arachidonic acid via cyclooxygenase (COX) enzymes and PGD2 synthases (PTDS). PGD2 is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. Structural and functional information about PGD2 can be found, e.g., in the entry for “Prostaglandin D2” of PubChem: pubchem.ncbi.nlm.nih.gov/compound/448457, the contents of which are herein incorporated by reference in their entirety.

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos, NP_000851.2, NP_001139288.1, NP_001243236.1, NP_001243234.1, NP_001243235.1, NP_001350503.1, NP_001243230.1) can be targeted, as can any isoform with 50%, 60%, 70%, 80%, 85%, 90%, 95%, or higher identity to the amino acid sequences of any of GenBank Accession Nos, NP_000851.2, NP_001139288.1, NP_001243236.1, NP_001243234.1, NP_001243235.1, NP_001350503.1, NP_001243230.1, or of any other 15-PGDH enzyme.

A “15-PGDH inhibitor” refers to any agent that is capable of inhibiting, reducing, decreasing, attenuating, abolishing, eliminating, slowing, and/or counteracting in any way any aspect of the expression, stability, and/or activity of 15-PGDH. A 15-PGDH inhibitor can, for example, reduce any aspect of the expression, e.g., transcription, RNA processing, RNA stability, and/or translation of a gene encoding 15-PGDH, e.g., the human HPGD gene, by, e.g., 10%, 15%, 20%, 25%, 30%, 35%, 40%, 45%, 50%, 55%, 60%, 65%. 70%, 75%, 80%, 85%, 90%, 95%, or more as compared to a control, e.g., in the absence of the inhibitor, in vitro or in vivo. Similarly, a 15-PGDH inhibitor can, for example, reduce the activity, e.g., enzymatic activity, of a 15-PGDH enzyme by, e.g., 10%, 15%, 20%, 25%. 30%. 35%. 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor can, for example, reduce the stability of a 15-PGDH enzyme by, e.g., 10%, 15%, 20%. 25%, 30%. 35%. 40%. 45%, 50%, 55%. 60%, 65%, 70%, 75%, 80%, 85%, 90%, 95%, or more as compared to a control, e.g., in the absence of the inhibitor, in vitro or in vivo. A “15-PGDH inhibitor”, also referred to herein as an “15-PGDH agent” or a “15-PGDH compound.” can be any molecule, either naturally occurring or synthetic, e.g., peptide, protein, oligopeptide (e.g., from about 5 to about 25 amino acids in length, e.g., about 5, about 10, about 15, about 20, or about 25 amino acids in length), small molecule (e.g., an organic molecule having a molecular weight of less than about 2500 daltons. e.g., less than about 2000, less than about 1000, or less than about 500 daltons), antibody, nanobody, polysaccharide, lipid, fatty acid, inhibitory RNA (e.g., siRNA, shRNA, microRNA), modified RNA, polynucleotide, oligonucleotide, e.g., antisense oligonucleotide, aptamer, affimer, drug compound, or other compound.

A “senolytic agent” refers to any agent that is capable of inducing the death of senescent cells, e.g., inducing the death of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of a population of senescent cells, in vitro or in vivo. A non-limiting list of senolytic agents that can be used in the present methods include Bcl2 inhibitors (e.g., navitoclax (ABT-263), ABT-737), pan-tyrosine kinase inhibitors (e.g., dasatinib), flavonoids (e.g., quercetin), peptides that interfere with the FOXO4-p53 interaction (e.g., FOXO4-DRI), a selective targeting system of senescent cells using galactooligosaccharide-coated nanoparticles, HSP90 inhibitors (e.g., 17-DMAG), and combinations thereof. In particular embodiments, a senolytic agent is capable of inducing the death of, e.g., 10%. 15%, 20%. 25%, 30%, 35%, 40%, 45%, 50%, 55%, 60%, 65%, 70%, 75%, 80%, 85%, 90%, 95% or more senescent cells, e.g., macrophages and/or fibroadipocytes within non-skeletal muscle tissue and/or organs.

The terms “expression” and “expressed” refer to the production of a transcriptional and/or translational product, e.g., of a nucleic acid sequence encoding a protein (e.g., 15-PGDH). In some embodiments, the term refers to the production of a transcriptional and/or translational product encoded by a gene (e.g., the human HPGD gene) or a portion thereof. The level of expression of a DNA molecule in a cell may be assessed on the basis of either the amount of corresponding mRNA that is present within the cell or the amount of protein encoded by that DNA produced by the cell.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a 15-PGDH protein). Examples of antibody-binding fragments include, but are not limited to, a Fab fragment (a monovalent fragment consisting of the VL, VH, CL, and CH1 domains), F(ab′)2 fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen (see, e.g., Fundamental Immunology (Paul ed., 4th ed. 2001).

The phrase “specifically binds” refers to a molecule (e.g., a 15-PGDH inhibitor such as a small molecule or antibody) that binds to a target with greater affinity, avidity, more readily, and/or with greater duration to that target in a sample than it binds to a non-target compound. In some embodiments, a molecule that specifically binds a target (e.g., 15-PGDH) binds to the target with at least 2-fold greater affinity than non-target compounds, e.g., at least 3-fold, at least 4-fold, at least 5-fold, at least 6-fold, at least 7-fold, at least 8-fold, at least 9-fold, at least 10-fold, at least 20-fold, at least 25-fold, at least 50-fold or greater affinity. For example, in some embodiments, a molecule that specifically binds to 15-PGDH typically binds to 15-PGDH with at least a 2-fold greater affinity than to a non-15-PGDH target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer”, “administering”, or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. In some cases, the administration is systemic administration (e.g., administration into the circulatory system such that multiple tissues and/or organs are treated or affected). In some cases, the administration is local administration (e.g., directly to the tissue or organ such that the tissue and/or organ is treated or affected). One skilled in the art will know of additional methods for administering a therapeutically effective amount of the compounds described herein.

The term “therapeutically effective amount” or “therapeutically effective dose” or “effective amount” refers to an amount of a compound (e.g., 15-PGDH inhibitor) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, and/or ongoing disease process and type of treatment desired (e.g., aggressive vs. conventional treatment). Therapeutically effective amounts of a compound (e.g., 15-PGDH inhibitor), as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutical composition” as used herein refers to a composition comprising a compound (e.g., 15-PGDH inhibitor) as described herein and one or more pharmaceutically acceptable carriers and/or pharmaceutically acceptable excipients.

The term “pharmaceutically acceptable carrier” as used herein refers to a carrier or a diluent that does not cause significant irritation to an organism and does not abrogate the biological activity and properties of the administered compound.

The terms “subject”, “individual”, and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “acute regimen”, in the context of administration of a compound, refers to a temporary or brief application of a compound to a subject, e.g., human subject, or to a repeated application of a compound to a subject, e.g., human subject, wherein a desired period of time (e.g., 1 day) lapses between applications. In some embodiments, an acute regimen includes an acute exposure (e.g., a single dose) of a compound to a subject over the course of treatment or over an extended period of time. In other embodiments, an acute regimen includes intermittent exposure (e.g., repeated doses) of a compound to a subject in which a desired period of time lapses between each exposure.

The term “chronic regimen,” in the context of administration of a compound, refers to a repeated, chronic application of a compound to a subject, e.g., human subject, over an extended period of time such that the amount or level of the compound is substantially constant over a selected time period. In some embodiments, a chronic regimen includes a continuous exposure of a compound to a subject over an extended period of time.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency. e.g., as described in Dar et al. (2016) Scientific Reports 6: article no. 20031 (2016), and as presented in the database accessible at crdd.osdd.net/servers/sirnamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the terms “identical” or percent “identity”, in the context of describing two or more polynucleotide or amino acid sequences, refer to two or more sequences or specified subsequences that are the same. Two sequences that are “substantially identical” have at least about 60% identity, preferably at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

The terms “rejuvenating” or “rejuvenation” as used herein refers to the restoration or revitalization of an individual, a cell, a tissue, and/or an organ (e.g., to a more “youthful” state, e.g., to more closely resemble a young individual, young cell, young tissue, and/or young organ). In some cases, the terms “rejuvenating” or “rejuvenation” may refer to increasing, enhancing, restoring, regenerating, and/or improving a function of a (e.g., aged) tissue and/or (e.g., aged) organ (e.g., to more closely resemble a function of a young tissue and/or young organ). In some cases, the terms “rejuvenating” or “rejuvenation” may refer to increasing, enhancing, restoring, regenerating, and/or improving a structure of a (e.g., aged) tissue and/or (e.g., aged) organ (e.g., to more closely resemble a structure of a young tissue and/or young organ). In some cases, the terms “rejuvenating” or “rejuvenation” may refer to decreasing levels of 15-PGDH and/or activity of 15-PGDH, and/or increasing levels of PGE2 and/or PGD2 in a (e.g., aged) tissue or (e.g., aged) organ (such that the levels of 15-PGDH and/or activity of 15-PGDH and/or levels of PGE2 and/or PGD2 more closely resemble those of young tissue or young organs). In some instances, the term “rejuvenating” or “rejuvenation” may refer to restoring or changing a transcriptome, a proteome, an epigenome, and/or a cellular composition of an aged cell, an aged tissue, or an aged organ to more closely resemble a transcriptome, a proteome, an epigenome, and/or a cellular composition of a young cell, a young tissue, or a young organ.

### 4. Methods of Rejuvenating Tissue and/or Organ Function in Subjects with Age-Related Conditions

In one embodiment, a method is provided for rejuvenating a function of an aged non-skeletal muscle tissue or aged non-skeletal muscle organ in an individual, the method comprising: administering to the individual or to the aged non-skeletal muscle tissue or aged non-skeletal muscle organ a 15-hydroxyprostaglandin dehydrogenase (15-PGDH) inhibitor in an amount effective to inhibit 15-PGDH activity and/or reduce 15-PGDH levels in the individual, thereby rejuvenating the function of the aged non-skeletal muscle tissue or the aged non-skeletal muscle organ in the individual.

Further provided herein is a method of enhancing a function of a non-skeletal muscle tissue or non-skeletal muscle organ in a subject, the method comprising: administering to the subject a 15-PGDH inhibitor in an amount effective to inhibit 15-PGDH activity and/or reduce 15-PGDH levels in the non-skeletal muscle tissue or non-skeletal muscle organ, thereby enhancing a function of the non-skeletal muscle tissue or non-skeletal muscle organ in the subject. In some cases, the subject is less than 30 years of age (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 years of age). In other cases, the subject is greater than 30 years of age.

In various aspects, the aged non-skeletal muscle tissue and/or organ may have one or more senescent cells (e.g., present within or near the aged non-skeletal muscle tissue and/or organ). In some cases, the aged non-skeletal muscle tissue and/or organ may have a plurality of senescent cells (e.g., present within or near the aged non-skeletal muscle tissue and/or organ). In some cases, the aged non-skeletal muscle tissue and/or organ may have an increased accumulation of senescent cells (e.g., within or near the aged non-skeletal muscle tissue and/or organ) (e.g., relative to young non-skeletal muscle tissue and/or organ). In some cases, the aged non-skeletal muscle tissue and/or organ may have a number of senescent cells that is higher (e.g., substantially higher) than a number typically found in young non-skeletal muscle tissue and/or organ. The senescent cells may express one or more senescent markers. The senescent cells may have an increased level of one or more senescent markers relative to a non-senescent cell. The one or more senescent markers may be, without limitation, p15Ink4b, p16Ink4a, p19Arf, p21, Mmp13, Il1a, Il1b, and Il6. In various aspects, the subject may be selected for treatment (e.g., by any method disclosed herein) based on a level of senescent cells present within the aged non-skeletal muscle tissue and/or organ and/or based on the presence or levels of one or more senescent markers. In some cases, the presence of senescent cells within the aged non-skeletal muscle tissue and/or organ (e.g., at a number higher than a number typically found in young non-skeletal muscle tissue and/or organ) and/or the presence and/or levels of one or more senescent markers may indicate that a treatment (e.g., any disclosed herein) is likely to provide a therapeutic benefit. In some cases, the senescent cells may express 15-PGDH (e.g., at levels effective to decrease a level of PGE2 within the aged non-skeletal muscle tissue and/or organ). In some cases, the senescent cells may be macrophages.

In various aspects, the subject may express one or more biomarkers of aging. A biomarker of aging may include, without limitation, an increase in 15-PGDH levels (e.g., relative to a level present in young non-skeletal muscle tissue and/or organ), a decrease in PGE2 levels (e.g., relative to a level present in young non-skeletal muscle tissue and/or organ), an increase in a PGE2 metabolite (e.g., relative to a level present in young non-skeletal muscle tissue and/or organ), an increase or a greater accumulation of senescent cells (e.g., relative to a level present in young non-skeletal muscle tissue and/or organ), an increase in expression of one or more atrogenes (e.g., Atrogin1 (MAFbx1), Fbxo30 (MuSA), and Trim63 (MuRF1)) (e.g., relative to a level present in young non-skeletal muscle tissue and/or organ), a decrease in mitochondria biogenesis and/or function (e.g., relative to a level present in young non-skeletal muscle tissue and/or organ), and an increase in transforming growth factor pathway signaling (e.g., an increase in expression of one or more genes involved in a transforming growth factor signaling pathway. e.g., one or more of Activin receptor, Myostatin, a SMAD protein, and a bone morphogenetic protein) (e.g., relative to a level present in young non-skeletal muscle tissue and/or organ). In some cases, a biomarker of aging may include increased levels or activity of 15-PGDH (e.g., within the aged non-skeletal muscle tissue and/or organ) (e.g., relative to a level present in young non-skeletal muscle tissue and/or organ). In some cases, a biomarker of aging may include decreased levels of PGE2 (e.g., within the aged non-skeletal muscle tissue and/or organ) (e.g., relative to a level present in young non-skeletal muscle tissue and/or organ). In some cases, a biomarker of aging may include increased levels of a PGE2 metabolite (e.g., 15-keto PGE2 and 13,14-dihydro-15-keto PGE2, e.g., within the aged non-skeletal muscle tissue and/or organ) (e.g., relative to a level present in young non-skeletal muscle tissue and/or organ). In some cases, the presence of a biomarker of aging may indicate that the subject is likely to benefit from treatment according to any method disclosed herein. Young non-skeletal muscle tissue and/or organs may include non-skeletal muscle tissue and/or organs from a subject under the age of 30 (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 years of age).

In various aspects, levels of PGE2 present within the aged non-skeletal muscle tissue and/or organ may be increased (e.g., after treatment with a 15-PGDH inhibitor, e.g., according to methods provided herein) relative to levels present in the aged non-skeletal muscle tissue and/or organ prior to the treatment (e.g., with the 15-PGDH inhibitor). PGE2 levels in the aged non-skeletal muscle tissue and/or organ may be increased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to levels present in the aged non-skeletal muscle tissue and/or organ prior to the treatment (e.g., with the 15-PGDH inhibitor). In various aspects, levels of PGE2 present within the aged non-skeletal muscle tissue and/or organ may be increased (e.g., after treatment with a 15-PGDH inhibitor, e.g., according to methods provided herein) to a level substantially similar to a level present in young non-skeletal muscle tissue and/or organ. PGE2 levels in the aged non-skeletal muscle tissue and/or organ may be increased (e.g., by any method disclosed herein) to a level within about 50% or less of a level present in young non-skeletal muscle tissue and/or organ (e.g., within about 40%, within about 35%, within about 30%, within about 25%, within about 20%, within about 15%, within about 10%, within about 5%, or within about 1%).

In various aspects, levels of PGE2 metabolites present within the aged non-skeletal muscle tissue and/or organ may be decreased (e.g., after treatment with a 15-PGDH inhibitor, e.g., according to methods provided herein) relative to levels present in the aged non-skeletal muscle tissue and/or organ prior to the treatment (e.g., with the 15-PGDH inhibitor). PGE2 metabolite levels in the aged non-skeletal muscle tissue and/or organ may be decreased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to levels present in the aged non-skeletal muscle tissue and/or organ prior to the treatment (e.g., with the 15-PGDH inhibitor). In various aspects, levels of PGE2 metabolites present within the aged non-skeletal muscle tissue and/or organ may be decreased (e.g., after treatment with a 15-PGDH inhibitor, e.g., according to methods provided herein) to a level substantially similar to a level present in young non-skeletal muscle tissue and/or organ. PGE2 metabolite levels in the aged non-skeletal muscle tissue and/or organ may be decreased (e.g., by any method disclosed herein) to a level within about 50% or less of a level present in young non-skeletal muscle tissue and/or organ (e.g., within about 40%, within about 35%, within about 30%, within about 25%, within about 20%, within about 15%, within about 10%, within about 5%, or within about 1%). The PGE2 metabolite may be 15-keto PGE2, 13,14-dihydro-15-keto PGE2, or both. The PGE2 metabolite may be 15-keto PGE2, 13,14-dihydro-15-keto PGE2, or both.

In various aspects, a function of the aged non-skeletal muscle tissue and/or organ may be enhanced (e.g., after treatment with a 15-PGDH inhibitor, e.g., according to methods provided herein) relative to the aged non-skeletal muscle tissue and/or organ prior to the treatment (e.g., with the 15-PGDH inhibitor). A function of the aged non-skeletal muscle tissue and/or organ may be enhanced (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to levels present in the aged non-skeletal muscle tissue and/or organ prior to the treatment (e.g., with the 15-PGDH inhibitor). In various aspects, a function of the aged non-skeletal muscle tissue and/or organ may be enhanced (e.g., after treatment with a 15-PGDH inhibitor, e.g., according to methods provided herein) to a level substantially similar to a level present in young non-skeletal muscle tissue and/or organ. A function of the aged non-skeletal muscle tissue and/or organ may be enhanced (e.g., by any method disclosed herein) to a level within about 50% or less of a level present in young non-skeletal muscle tissue and/or organ (e.g., within about 40%, within about 35%, within about 30%, within about 25%, within about 20%, within about 15%, within about 10%, within about 5%, or within about 1%). A function may include increased protein synthesis, increased cell proliferation, increased cell survival, decreased protein degradation, or any combination thereof. A function may include any function disclosed herein.

In some instances, treatment (e.g., with a 15-PGDH inhibitor, e.g., according to methods provided herein) may result in rejuvenation of the aged non-skeletal muscle tissue and/or organ (e.g., an increase in one or more functions of the aged non-skeletal muscle tissue) such that the aged non-skeletal muscle tissue and/or organ more closely resembles young non-skeletal muscle tissue and/or organs.

The present disclosure provides methods of increasing the function, health, and other properties of non-skeletal muscle tissues and/or organs in subjects, e.g., human subjects, with an age-related condition or disease, or due to the natural aging process, comprising administering a 15-PGDH inhibitor to the subject. The administration of the 15-PGDH inhibitor can be systemic or local, and can enhance any of a number of aspects of the tissue and/or organ including enhancing function, enhancing physiological activity, enhancing endurance, and/or enhancing performance on any assay for assessing tissue or organ function, or any other measure of tissue or organ function or health in the subject. In some embodiments, the administration of the 15-PGDH inhibitor results in protection against cell death in the non-skeletal muscle tissue and/or organ in the subject. In some embodiments, the administration of the 15-PGDH inhibitor results in reduced protein degradation in the non-skeletal muscle tissue and/or organ in the subject. In some embodiments, the administration of the 15-PGDH inhibitor results in increased protein synthesis in the non-skeletal muscle tissue and/or organ in the subject. In some embodiments, administration of the 15-PGDH inhibitor may result in increased endurance (e.g., during exercise, e.g., as measured on a treadmill). In some cases, the increased endurance of the subject (e.g., during exercise) may be due to an increased function and/or rejuvenation of the aged non-skeletal muscle tissue and/or tissue (e.g., heart, lungs, bones, etc.).

The present disclosure also provides methods of measuring 15-PGDH levels in non-skeletal muscle tissues and/or organs of a subject with an age-related condition or an aged subject. Such methods are useful, e.g., for the use of 15-PGDH as a biomarker of aging or aging non-skeletal muscle tissues and/or organs and/or for a loss or decrease of function of non-skeletal muscle tissues and/or organs, e.g., wherein an elevated level of 15-PDGH levels or activity, e.g., an increase of about 10%, about 20%, about 30%, about 40%, about 50%, about 60%, about 70%, about 80%, about 90%, about 100% or more relative to a control level in a subject without an age-related condition is indicative of aging or a loss or decrease of function in the tissue and/or organ. In such methods, 15-PGDH can be assessed in any of a number of ways, e.g., by detecting levels of a transcript encoding a 15-PGDH protein, by detecting levels of a 15-PGDH polypeptide, or by detecting 15-PGDH enzymatic activity.

In particular embodiments, the inhibition of 15-PGDH in the subject leads to an increase in PGE2 and/or PGD2, e.g., an elevation, increase, or restoration of PGE2 and/or PGD2 levels, in the non-skeletal muscle tissue and/or organ of the subject, and a decrease in PGE2 and/or PGD2 metabolites such as 15-keto-PGE2, 13,14-dihydro-15-keto-PGE2 (PGEM), 15-keto-PGD2, and 13,14-Dihydro-15-keto-PGD2. In some embodiments, the inhibition also leads to increased signaling through PGE2 receptors, e.g., EP1, EP2, EP3, and/or EP4 (also known as Ptger1, Ptger2, Ptger3, Ptger4) in the non-skeletal muscle tissue and/or organ. In some embodiments, the inhibition also leads to increased signaling through PGD2 receptors, e.g., DP1 and/or DP2 (also known as PTGDR1, PTGDR2/CRTH2).

In particular embodiments, the herein-described benefits of 15-PGDH inhibitor administration in the non-skeletal muscle tissue and/or organ, e.g., enhanced tissue health, function, physiological activity, etc., occur independently of any regeneration of the tissue and/or organ in the subject. In other words, while there may be regeneration of the tissue and/or organ in the subject, e.g., if the tissue and/or organ has been injured or damaged, the herein-described effects do not require the regeneration and would occur even without the regeneration. In particular embodiments, the non-skeletal muscle tissue and/or organ is not injured or damaged and has not or does not undergo regeneration.

In some embodiments, the administration of the 15-PGDH inhibitor inhibits 15-PGDH activity or reduced 15-PGDH levels in senescent cells, e.g., macrophages, fibroadipocytes, other mononuclear interstitial tissue resident cells including other immune cells, fibroblasts, endothelial cells, preadipocytes, and/or adipocytes, within the non-skeletal muscle tissue and/or organ of the subject. Consequently, the administration of the 15-PGDH inhibitor may result in decreased fibrosis or may inhibit fibrosis in various aged tissues and/or organs (e.g., heart, lung, etc.). In some embodiments, the methods further comprise the administration of a senolytic agent to the subject. Examples of senolytic agents that can be used include, inter alia, Bcl2 inhibitors such as navitoclax (also known as ABT-263) and ABT-737, pan-tyrosine kinase inhibitors such as dasatinib together with a flavonoid such as quercetin, a peptide which interferes with the FOXO4-p53 interaction such as FOXO4-DRI, a selective targeting system of senescent cells using galactooligosaccharides-coated nanoparticles, a combination drug therapy comprising dasatinib and quercetin, and HSP90 inhibitors such as 17-DMAG. It will be appreciated that the senolytic agent can be administered together with the 15-PGDH inhibitor, e.g., within a single pharmaceutical formulation, or separately.

### Rejuvenating Aged Spleen

The spleen is a secondary lymphoid organ consisting of two compartments—the blood-containing red pulp region where pathogens and aged erythrocytes are removed by macrophages and the white pulp region comprising of B and T cells responsible for the adaptive immune response. The white pulp region is surrounded by a marginal zone which is involved in innate and adaptive immunity. It is comprised of stromal cells associated with a subset of macrophages and B cells that enable the capture of blood borne antigens.

The spleen's main function is to filter blood. For example, the spleen filters old, damaged, or abnormal red blood cells (erythrocytes) from the blood. The spleen also filters microorganisms and pathogens from the blood, as well as cellular debris. The spleen also can store leftover used products including iron, which is stored in the form of ferritin or bilirubin and returned to the bone marrow for hemoglobin production. The spleen also can store blood and can release the blood in the case of excessive blood loss. The spleen is also responsible for maturation of lymphoid cell types involved in the adaptive immune response. The spleen is also involved in initiating immune reactions to blood-borne antigens.

During the aging process, one or more of these functions may be abrogated or reduced. In elderly people, splenic function has been shown to be decreased or impaired. In addition, aged spleens have been shown to exhibit abnormal morphology with aberrant lymphoid follicular structure when compared with young spleens. Aged spleens have been shown to exhibit a loss of the marginal zone which separates the germinal center from the red pulp area of the spleen. Aged splenic follicles have been shown to exhibit reduced cell density with areas of low cell-cell contact.

As depicted in Example 1, 15-PGDH levels are elevated in aged spleen relative to young spleen. Additionally, as depicted in Example 2, treatment with a 15-PGDH inhibitor rejuvenated aged splenic morphology. Accordingly, in one aspect of the disclosure, a method is provided for rejuvenating aged spleen by administering to an individual (e.g., an aged individual, e.g., having an aged spleen) or to an aged spleen a 15-PGDH inhibitor in an amount effective to inhibit 15-PGDH activity and/or to reduce 15-PGDH levels in the individual, thereby rejuvenating the aged spleen. In some cases, the administering is systemic administration (e.g., orally). In other cases, the administering is local administration (e.g., directly to the spleen, or to the central nervous system). In other cases, the administering is topical administration on the skin (e.g., as a cream or salve mixed with a carrier that allows the 15-PGDH inhibitor to penetrate through layers of the skin). Any suitable administration route as described herein may be used.

In various aspects, after administration of the 15-PGDH inhibitor, a function of the aged spleen is rejuvenated relative to a function of the aged spleen prior to treatment with the 15-PGDH inhibitor. In various aspects, after administration of the 15-PGDH inhibitor, a function of the aged spleen is rejuvenated to a level substantially similar to a level found in young spleen.

In some embodiments, after administration of the 15-PGDH inhibitor, the aged spleen exhibits increased or enhanced clearance of pathogens, microorganisms, cellular debris, and/or aged erythrocytes from the blood (e.g., as compared to the aged spleen prior to treatment with the 15-PGDH inhibitor) (e.g., as measured by changes to the hematocrit or number of white blood cells from a complete blood count, or by imaging for spleen size (e.g., ultrasound, MRI). In some embodiments, after administration of the 15-PGDH inhibitor, the aged spleen is capable of clearing pathogens, microorganisms, cellular debris, and/or aged erythrocytes from the blood at levels that are substantially similar to levels found in young spleen. In some embodiments, after administration of the 15-PGDH inhibitor, the aged spleen exhibits increased lymphoid maturation (e.g., as compared to the aged spleen prior to treatment with the 15-PGDH inhibitor) (e.g., as measured by antibody production against specific pathogens or microorganisms; or as measured by time to resolution of infection or symptoms after an infection by a virus or a microorganism; or as measured by reduced production of auto-reactive antibodies). In some embodiments, after administration of the 15-PGDH inhibitor, the aged spleen exhibits levels of lymphoid maturation that are substantially similar to levels found in young spleen. In some embodiments, after administration of the 15-PGDH inhibitor, the aged spleen exhibits increased antibody production (e.g., as compared to the aged spleen prior to treatment with the 15-PGDH inhibitor; e.g., to levels that are substantially similar to levels found in young spleen).

### Rejuvenating Aged Epithelial Tissue

The epithelial tissue lines the outer surfaces of organs and blood vessels throughout the body and the inner surfaces of cavities in many internal organs and is comprised of cell types of squamous, cuboidal, columnar, and pseudostratified. Squamous cells decrease friction of a surface for which fluids can move easily. Squamous epithelium can be found lining surfaces such as skin or alveoli in the lung, allowing passive diffusion of molecules or biomolecules. In some cases, squamous epithelium can also form the lining of cavities such as in blood vessels as endothelium, in the pericardium as mesothelium, or in other body cavities. Cuboidal epithelium can be found in secretive tissue such as the exocrine glands, in absorptive tissue such as the pancreas, the lining of the kidney tubules, in the ducts of the glands, covering the female ovary, or lining the walls of the seminiferous tubules in the testes. Cuboidal cells provide protection and can be active in pumping material in or out of the lumen or allowing passive diffusion, depending on location and specialization of the cuboidal cells. Simple cuboidal epithelium can differentiate to form the secretory and duct portions of glands. Stratified cuboidal epithelium protects areas such as the ducts of sweat glands, mammary glands, or salivary glands. Columnar epithelium forms the lining of the stomach and intestines and can possess microvilli for maximizing the surface area for absorption. These microvilli can form a brush border. Other columnar cells can be ciliated to move mucus in the function of mucociliary clearance. Other columnar cells can be ciliated and found in the fallopian tubes, the uterus, or central canal of the spinal cord. Some columnar cells can be specialized for sensory reception such as in the nose, ears, or the taste buds. Hair cells in the inner ears have stereocilia which are similar to microvilli. Goblet cells are modified columnar cells and are found between the columnar epithelial cells of the duodenum for secreting mucus as a lubricant. Single-layered non-ciliated columnar epithelium can indicate an absorptive function. Stratified columnar epithelium can be found in lobar ducts in the salivary glands, the eye, the pharynx, or sex organs. Pseudostratified epithelial cells can be ciliated, where the cilia are capable of energy-dependent pulsatile beating in a certain direction through interaction of cytoskeletal microtubules and connecting structural proteins and enzymes. In the respiratory tract, the wafting effect produced causes mucus secreted locally by the goblet cells (to lubricate and to trap pathogens and particles) to flow in that direction (typically out of the body). Ciliated epithelium can be found in the airways (nose, bronchi), but can also be found in the uterus and Fallopian tubes, where the cilia propel the ovum to the uterus.

The epithelial tissue's main functions include secretion, selective absorption, protection, transcellular transport, and/or sensing. For example, the epithelial tissue provides a protective barrier against mechanical, thermal, and physical injury and hazardous substances, prevents loss of moisture, reduces harmful effects of UV radiation, and/or acts as a sensory organ (touching, detecting temperature, etc.). During the aging process, one or more of these functions may be abrogated or reduced. In elderly people, epithelial tissue function has been shown to be decreased or impaired. In addition, aged epithelial tissue has been shown to exhibit abnormal morphology with flattened dermal-epidermal junction, giving the appearance of atrophy and cellular heterogeneity. The melanocyte density declines slowly, and the Langerhans cells decrease in number with advancing age. Among the dermal changes can be attenuation in the number and diameter of elastic fibers in the papillary dermis, an increase in number and thickness of the same fibers in the reticular dermis, and a coarsening of collagen fibers with an increase in density of the collagen network. A decrease in the dermal cell population as well as a functional decline in glandular activity are also noted with intrinsic aging. A decline in hair number, rate of growth, and diameter, along with a slowing of the rate of growth of nails, have also been associated with aging epithelial tissue.

In one aspect of the disclosure, a method is provided for rejuvenating aged epithelial tissue by administering to an individual (e.g., an aged individual, e.g., having an aged epithelial tissue) or to an aged epithelial tissue a 15-PGDH inhibitor in an amount effective to inhibit 15-PGDH activity and/or to reduce 15-PGDH levels in the individual, thereby rejuvenating the aged epithelial tissue. In some cases, the administering is systemic administration (e.g., orally or intravenously). In other cases, the administering is local administration (e.g., topically or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged epithelial tissue is rejuvenated relative to a function of the aged epithelial tissue prior to treatment with the 15-PGDH inhibitor. In various aspects, after administration of the 15-PGDH inhibitor, a function of the aged epithelial tissue is rejuvenated to a level substantially similar to a level found in young epithelial tissue.

In some embodiments, after administration of the 15-PGDH inhibitor, the aged epithelial tissue exhibits increased or enhanced skin functions such as secretion, selective absorption, protection, transcellular transport, and sensing (e.g., as compared to the aged epithelial tissue prior to treatment with the 15-PGDH inhibitor) as determined by measuring, e.g., collagen composition or abundance in the epithelial tissue, transepidermal water loss, epithelial tissue hydration (e.g., skin hydration), epithelial tissue dryness (e.g., skin dryness), epithelial tissue elasticity (e.g., skin elasticity), corneocyte adhesion, ceramide concentration, pruritus, water-holding capacity, epithelial skin smoothness and roughness (e.g., skin smoothness and roughness), epithelial tissue wrinkles (e.g., skin wrinkles), epithelial tissue scaling (e.g., skin scaling), epithelial tissue tightness or softness (skin tightness or softness), epithelial tissue reddening and erythema formation (e.g., skin reddening and erythema formation), capillary blood flow, protection of the epithelial tissue against oxidative (including UV-induced or non-UV-induced) damage to nucleic acid, lipid, or protein, depletion of Langerhans cells after UV exposure, delayed-type hypersensitivity immune response to recall antigens in epithelial tissue such as skin or epithelial tissue or skin sebum. In some embodiments, after administration of the 15-PGDH inhibitor, the aged epithelial tissue is capable of secretion, selective absorption, protection, transcellular transport, and sensing at levels that are substantially similar to levels found in young epithelial tissue. In some embodiments, after administration of the 15-PGDH inhibitor, the aged epithelial tissue exhibits enhanced skin, increased barrier function, increased hair growth, increased elasticity of skin, and stimulation of hair follicle stem cells. In some embodiments, after the administering, the epithelial tissue exhibits increased dermal thickness.

### Rejuvenating Aged Vascular Tissue

The vascular tissue is part of an organ system that permits blood to circulate and transport nutrients (such as amino acids and electrolytes), oxygen, carbon dioxide, hormones, or blood cells to and from the cells in the body to provide nourishment, immunity, and homeostasis such as stabilizing temperature or pH. Vascular tissue can include lymphatic system and cardiovascular system and comprises cell types of endothelial cells (ECs) and vascular smooth muscle cells (VSMCs).

The vascular tissue's main functions include transportation and passage of lymph or blood, which includes plasma, red blood cells, white blood cells, and platelets that are circulated by the heart through the vertebrate vascular system, carrying oxygen and nutrients to and waste materials away from all body tissues. Lymph can recycle excess blood plasma after it has been filtered from the interstitial fluid (between cells) and returned to the lymphatic system. During the aging process, one or more of these functions may be abrogated or reduced. In elderly people, vascular tissue function has been shown to be decreased or impaired. In addition, aged vasculature tissue has been shown to exhibit oxidative stress, mitochondrial dysfunction, impaired resistance to molecular stressors, chronic low-grade inflammation, genomic instability, cellular senescence, epigenetic alterations, loss of protein homeostasis, deregulated nutrient sensing, and/or stem cell dysfunction.

In one aspect of the disclosure, a method is provided for rejuvenating aged vascular tissue by administering to an individual (e.g., an aged individual, e.g., having an aged vascular tissue) or to an aged vascular tissue a 15-PGDH inhibitor in an amount effective to inhibit 15-PGDH activity and/or to reduce 15-PGDH levels in the individual, thereby rejuvenating the aged vascular tissue. In some cases, the administering is systemic administration (e.g., orally or intravenously). In other cases, the administering is local administration (e.g., intramuscularly or intravenously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged vascular tissue is rejuvenated relative to a function of the aged vascular tissue prior to treatment with the 15-PGDH inhibitor. In various aspects, after administration of the 15-PGDH inhibitor, a function of the aged vascular tissue is rejuvenated to a level substantially similar to a level found in young vascular tissue.

In some embodiments, after administration of the 15-PGDH inhibitor, the aged vascular tissue exhibits increased or enhanced vascular tissue functions (e.g., as compared to the aged vascular tissue prior to treatment with the 15-PGDH inhibitor) as determined by, e.g., electrocardiography, ultrasound imaging. X-ray computed tomography and positron emission tomography, magnetic resonance imaging, angiography, contrast enhanced ultrasound, optical coherence tomography, flow-sensitive 4D-magnetic resonance imaging, bright field microscopy, fluorescence microscopy, mathematical modeling and abdominal aortic aneurysms, tissue material properties and tensile testing, tissue elasticity imaging, atomic force microscopy, flow cytometry, microfluidics, micropipette aspiration, optical microscopy, optical tweezers, or electron microscopy. In some embodiments, after administration of the 15-PGDH inhibitor, the aged vascular tissue is capable of functioning at substantially similar to levels found in young vascular tissue.

### Rejuvenating Dental Tissue/Teeth

Teeth comprise several layers of hard and soft tissue. The outermost layer comprises enamel which comprises predominately inorganic minerals (e.g., hydroxyapatite). This outermost layer protects internal layers comprising dentin, cementum, and dental pulp. Dentin comprises hard tissue comprising minerals, organic materials, and water. Cementum comprises hard tissue comprising minerals, organic materials, and water which connects a tooth to the surrounding bone of a jaw. These hard tissues surround the interior dental pulp which comprises soft connective tissue.

During the aging process, changes in the relative abundance and distribution of these tissues take place. Old teeth display a smaller dental pulp chamber and compensatory thickening of the dentin and cementum. Furthermore, receding gums are common in aged individuals. Receding gums can expose the roots of teeth to inflammation and bacterial infection.

Accordingly, in one aspect of the disclosure, a method is provided for rejuvenating aged teeth and/or gums by administering to an individual (e.g., an aged individual, e.g., having aged teeth) or to aged teeth and/or gums a 15-PGDH inhibitor in an amount effective to inhibit 15-PGDH activity and/or to reduce 15-PGDH levels in the individual, thereby rejuvenating the aged teeth and/or gums. In some cases, the administering is systemic administration (e.g., orally). In other cases, the administering is local topical administration (e.g., directly to the oral cavity or buccal administration). Any suitable administration route as described herein may be used.

In various aspects, after administration of the 15-PGDH inhibitor, a property of the dental tissue is rejuvenated relative to a property of the aged dental tissue prior to treatment with the 15-PGDH inhibitor. In various aspects, after administration of the 15-PGDH inhibitor, a property of the aged dental tissue is rejuvenated to a level substantially similar to a level found in young dental tissue.

In some embodiments, after administration of the 15-PGDH inhibitor, the aged dental tissue displays an increased ratio of dentin to dental pulp (e.g., as compared to the aged dental tissue prior to treatment with the 15-PGDH inhibitor) (e.g., as measured by radiography of dental tissue). In some embodiments, after administration of the 15-PGDH inhibitor, the aged dental tissue displays a reduced level or reversal of the conversion of dental pulp to dentin compared to untreated aged dental tissue.

### Rejuvenating Aged Liver

The liver is a central metabolic and endocrine organ. Liver tissue comprises mostly hepatocytes in addition to liver sinusoidal endothelial cells (LSECs), hepatic stellate cells (HSCs) and Küpffer cells (KCs). Hepatocytes are responsible for the majority of hepatic functions.

The liver is responsible for maintaining whole-body homeostasis through regulation of metabolism, xenobiotic, and endobiotic clearance, and molecular biosynthesis. Specific functions of the liver include formation and excretion of bile, regulation of carbohydrate homeostasis, lipid synthesis and secretion of plasma lipoproteins, and control of cholesterol metabolism. The liver is also a central hub of metabolism, participating in the formation of urea, serum albumin, clotting factors, enzymes, and many other proteins.

During the aging process, one or more of these functions may be abrogated or reduced. In elderly people, liver mass and function has been shown to be decreased or impaired. This reduction in liver mass and function can lead to an impaired ability to metabolize many substances in older individuals. Additionally, hepatocytes in elderly individuals display denser body compartments than to hepatocytes in younger subjects.

Accordingly, in one aspect of the disclosure, a method is provided for rejuvenating aged liver by administering to an individual (e.g., an aged individual, e.g., having an aged liver) or to an aged liver a 15-PGDH inhibitor in an amount effective to inhibit 15-PGDH activity and/or to reduce 15-PGDH levels in the individual, thereby rejuvenating the aged liver. In some cases, the administering is systemic administration (e.g., orally). In other cases, the administering is local administration (e.g., directly to the liver). Any suitable administration route as described herein may be used.

In various aspects, after administration of the 15-PGDH inhibitor, a function of the aged liver is rejuvenated relative to a function of the aged liver prior to treatment with the 15-PGDH inhibitor. In various aspects, after administration of the 15-PGDH inhibitor, a function of the aged liver is rejuvenated to a level substantially similar to a level found in young liver.

In some embodiments, after administration of the 15-PGDH inhibitor, the aged liver exhibits increased mass or activity (e.g., as compared to the aged liver prior to treatment with 15-PGDH) (e.g., as measured by liver percussion or medical imaging). In some embodiments, after administration of the 15-PGDH inhibitor, the aged liver exhibits decreased or reversed reduction in size relative to the aged liver prior to treatment. In some embodiments, after administration of the 15-PGDH inhibitor, the aged liver exhibits increased xenobiotic clearance (e.g., as compared to the aged liver prior to treatment with the 15-PGDH inhibitor) (e.g., as measured by administering an exogenous substance to the individual and measuring metabolite formation and/or renal excretion). In some embodiments, after administration of the 15-PGDH inhibitor, the aged liver exhibits levels of xenobiotic clearance that are substantially similar to levels found in young liver. In some embodiments, after administration of the 15-PGDH inhibitor, the aged liver exhibits reduced levels of fibrosis. In some embodiments, after administration of the 15-PGDH inhibitor, the liver exhibits decreased fatty acid storage and/or exhibits decreased liver adipose content.

### Rejuvenating Aged Hair

Hair is a proteinaceous that grows from the follicles found in the dermis. Hair may be characterized by its color, quantity, and quality. Specifically, hair may be characterized by the presence or absence of pigment (i.e., graying), thickness and curvature, as well as changes in the amount of hair over time (i.e., hair loss).

During the aging process, one or more of these properties may be abrogated or reduced. Changes in hair production may lead to hair loss. Additionally, properties of the hair fiber in aged hair differs from young hair. Aged hair may differ in pigmentation (graying), diameter, curvature, structural properties (stretching, bending, torsional rigidity), and lipid composition.

Accordingly, in one aspect of the disclosure, a method is provided for rejuvenating aged hair by administering to an individual (e.g., an aged individual, e.g., having aged hair) or to aged hair a 15-PGDH inhibitor in an amount effective to inhibit 15-PGDH activity and/or to reduce 15-PGDH levels in the individual, thereby rejuvenating the aged hair. In some cases, the administering is systemic administration (e.g., orally). In other cases, the administering is topical administration (e.g., directly to the hair or scalp). Any suitable administration route as described herein may be used.

In various aspects, after administration of the 15-PGDH inhibitor, a property of the aged hair is rejuvenated relative to a property of the aged hair prior to treatment with the 15-PGDH inhibitor. In various aspects, after administration of the 15-PGDH inhibitor, a property of the aged hair is rejuvenated to a level substantially similar to a level found in young hair.

In some embodiments, after administration of the 15-PGDH inhibitor, the aged hair displays a larger diameter (e.g., as compared to the aged hair prior to treatment with the 15-PGDH inhibitor) (e.g., as measured by visual inspection, microscopy, or diffraction of a laser). In some embodiments, after administration of the 15-PGDH inhibitor, the individual exhibits less or no hair loss. In some embodiments, after administration of the 15-PGDH inhibitor, the aged hair exhibits levels of pigmentation that are substantially similar to young hair. In some embodiments, pattern baldness is reversed.

### Rejuvenating Aged Small Intestine

The small intestine is a principal organ of the digestive tract. The small intestine comprises three main regions—the duodenum, the jejunum, and the ileum. The duodenum is the shortest and prepares gastric chyme for transit through the rest of the small intestine and absorption through the villi. The jejunum is where products of digestion are principally absorbed through the villi. The ileum is the final section of the small intestine and is where vitamin B12 and bile acids as well as any remaining nutrients are absorbed.

The main function of the small intestine is to absorb nutrients from food. Most of chemical digestion takes place in the small intestine. Once nutrients are degraded into smaller molecules, they are absorbed through the villi which line the length of the small intestine. The small intestine also hosts gut flora which further aid in digestion of certain nutrients and contribute to immunity.

During the aging process, one or more of these functions may be abrogated or reduced. In elderly people, production of the enzyme lactase decreases which can lead to difficulty digesting dairy products. Additionally, changes in the gut flora comprising excessive growth of certain bacteria have been shown to occur with age that lead to difficulty absorbing certain nutrients such as calcium, folic acid, vitamin B12, and iron.

Accordingly, in one aspect of the disclosure, a method is provided for rejuvenating aged small intestine by administering to an individual (e.g., an aged individual, e.g., having an aged small intestine) or to an aged small intestine a 15-PGDH inhibitor in an amount effective to inhibit 15-PGDH activity and/or to reduce 15-PGDH levels in the individual, thereby rejuvenating the small intestine. In some cases, the administering is systemic administration (e.g., orally). In other cases, the administering is local administration (e.g., directly to the small intestine). Any suitable administration route as described herein may be used.

In various aspects, after administration of the 15-PGDH inhibitor, a function of the aged small intestine is rejuvenated relative to a function of the aged small intestine prior to treatment with the 15-PGDH inhibitor. In various aspects, after administration of the 15-PGDH inhibitor, a function of the aged small intestine is rejuvenated to a level substantially similar to a level found in young small intestine.

In some embodiments, after administration of the 15-PGDH inhibitor, the aged small intestine exhibits increased production of lactase (e.g., as compared to the aged spleen prior to treatment with the 15-PGDH inhibitor) (e.g., as measured by a lactose tolerance test). In some embodiments, after administration of the 15-PGDH inhibitor, the aged small intestine is capable of producing lactase at levels that are substantially similar to levels found in young small intestine. In some embodiments, after administration of the 15-PGDH inhibitor, the aged small intestine exhibits reduced growth of certain bacteria (e.g., as compared to the aged small intestine prior to treatment with the 15-PGDH inhibitor) (e.g., as measured by breath tests, medical imaging, or intestinal fluid culture, or by measuring levels of calcium, iron, folic acid, or vitamin B12 in a sample from the individual). In some embodiments, after administration of the 15-PGDH inhibitor, the aged small intestine exhibits gut flora substantially similar to that of a young small intestine.

### Rejuvenating Aged Colon

The colon is the largest component of the large intestine which is the last part of the digestive track. The large intestine comprises the cecum and appendix, the ascending colon, the transverse colon, the descending colon, the sigmoid colon, and the rectum.

The colon's main function is to complete the digestive process by absorbing water and salt from solid wastes before they are eliminated from the body. The movement of contents through the colon is accomplished by peristalsis. Additionally, the colon and large intestine comprise gut flora which can further digest some material not otherwise digested by the digestive track.

During the aging process, one or more of these functions may be abrogated or reduced. In elderly people, the movement of material through the colon via peristalsis has been shown to be decreased. This decrease in peristalsis can lead to constipation and reduce the ability of the colon to perform the function of absorbing water from solid wastes.

Accordingly, in one aspect of the disclosure, a method is provided for rejuvenating aged colon by administering to an individual (e.g., an aged individual, e.g., having an aged colon) or to an aged colon a 15-PGDH inhibitor in an amount effective to inhibit 15-PGDH activity and/or to reduce 15-PGDH levels in the individual, thereby rejuvenating the aged colon. In some cases, the administering is systemic administration (e.g., orally). In other cases, the administering is local administration (e.g., directly to the colon). Any suitable administration route as described herein may be used.

In various aspects, after administration of the 15-PGDH inhibitor, a function of the aged colon is rejuvenated relative to a function of the aged colon prior to treatment with the 15-PGDH inhibitor. In various aspects, after administration of the 1-PGDH inhibitor, a function of the aged colon is rejuvenated to a level substantially similar to a level found in young colon.

In some embodiments, after administration of the 15-PGDH inhibitor, the aged colon exhibits increased or enhanced peristalsis (e.g., as compared to the aged colon prior to treatment with the 15-PGDH inhibitor) (e.g., as measured by a physician during a general physical exam, a digital rectal exam, blood tests, x-ray, colonic transit study, colonoscopy, sigmoidoscopy, or the like). In some embodiments, after administration of the 15-PGDH inhibitor, the aged colon is exhibits peristalsis at levels that are substantially similar to a young colon or improved recovery from ulcerative colitis including diarrhea and gastrointestinal bleeding.

### Rejuvenating Aged Ovaries and Other Reproductive Tissues

Ovaries comprise the female gonads. The outer layer of the ovaries is the ovarian cortex comprising ovarian follicles suspended in a matrix of stromal cells. In addition to the ovaries, key reproductive tissues include the oviduct and the uterus. The oviduct is the site for transport of oocytes and the platform for fertilization and early embryo development. The uterus is the essential organ for pregnancy that provides the structure for the development of an embryo.

The main function of the ovaries are the produce and release oocytes. The ovaries are also involved in the production and secretion of hormones such as estrogen, androgen, inhibin, and progesterone and therefore in the regulation of pregnancy and secondary sex characteristics. The oviduct and uterus are also involved in the development of an embryo throughout pregnancy.

During the aging process, one or more of these functions may be abrogated or reduced. In elderly people, a decline in the number of ovary follicles leads to a decline in the performance of the ovaries comprising a decrease in the number of oocytes. In addition, there is a parallel increase in pregnancy failure and chromosomally aberrant conceptions with increasing age.

Accordingly, in one aspect of the present disclosure, a method is provided for rejuvenating aged ovaries and other reproductive tissues by administering to an individual (e.g., an aged individual, e.g., having aged ovaries or other reproductive tissues) or to aged ovaries or other reproductive tissues a 15-PGDH inhibitor in an amount effective to inhibit 15-PGDH activity and/or to reduce 15-PGDH levels in the individual, thereby rejuvenating the aged ovaries or other reproductive tissues. In some cases, the administering is system administration (e.g., orally). In other cases, the administering is local administration (e.g., directly to the reproductive tissues). Any suitable administration route as described herein may be used.

In various aspects, after administration of the 15-PGDH inhibitor, the aged ovaries or other reproductive tissues is rejuvenated relative to a function of the aged reproductive tissues prior to treatment with the 15-PGDH inhibitor. In various aspects, after administration of the 15-PGDH inhibitor, a function of the aged reproductive tissues is rejuvenated to a level substantially similar to a level found in young reproductive tissues.

In some embodiments, after administration of the 15-PGDH inhibitor, the aged reproductive tissues exhibit reduced or halted ovary decline (e.g., as compared to the aged reproductive tissues prior to treatment with the 15-PGDH) (e.g., as measured by blood or urine tests for hormone levels or egg reserves or imaging tests and procedures such as ultrasound exam, sonohysterography, hysterosalpingoraphy, hysteroscopy, or laparoscopy). In some embodiments, after administration of the 15-PGDH inhibitor, the aged ovaries show stopped or reduced decline in the number of ovary follicles or ovary follicles substantially similar to young ovaries. In some embodiments, after administration of the 15-PGDH inhibitor, the individual exhibits a reduction in pregnancy failure and/or number of chromosomally aberrant conceptions (e.g., as compared to the individual prior to treatment with the 15-PGDH inhibitor) (e.g., as measured by amniocentesis or chorionic villus sampling). In some embodiments, after administration of the 15-PGDH inhibitor, the individual exhibits a rate of pregnancy failure and/or chromosomally aberrant conceptions that is substantially similar to that of an individual with young ovaries and other reproductive tissues.

### Rejuvenating Aged Skin/Epidermal Tissue

The epidermal tissue (or skin) serves as a barrier to protect the body against microbial pathogens, oxidant stress (UV light), and chemical compounds, and provides mechanical resistance to minor injury. The epidermal tissue can contain nerve receptors that allow a subject to feel touch, pain, and pressure, help control fluid and electrolyte balance, help control body temperature, and/or protect a subject from the environment. The epidermal tissue can be divided into three main parts. The outer part (or epidermis) can contain skin cells, pigment, and proteins. The middle part (or dermis) contains skin cells, blood vessels, nerves, hair follicles, and oil glands. The dermis can provide nutrients to the epidermis. The inner layer under the dermis (or the subcutaneous layer) can contain sweat glands, some hair follicles, blood vessels, and fat. Each layer can also contain connective tissue with collagen fibers to give support and elastin fibers to provide flexibility and strength.

With aging, the outer skin layer (epidermis) thins. The number of cell layers may remain unchanged. The number of pigment-containing cells (melanocytes) can decrease. The remaining melanocytes can increase in size. Aging skin may look thinner, paler, and clear (translucent). Pigmented spots including age spots or “liver spots” may appear in sun-exposed areas. The medical term for these areas is lentigos. Changes in the connective tissue can reduce the skin's strength and elasticity (known as elastosis). The blood vessels of the dermis can become more fragile, which may lead to bruising, bleeding under the skin (often called senile purpura), cherry angiomas, and similar conditions. Sebaceous glands can produce less oil as one ages. This can make it harder to keep the skin moist, resulting in dryness and itchiness. The subcutaneous fat layer may thin so it has less insulation and padding, which may increase risk of skin injury and reduce ability to maintain body temperature. Some medicines may be absorbed by the fat layer. Shrinkage of this layer may change the way that these medicines work. The sweat glands can produce less sweat during aging, which can increase risk for overheating or developing heat stroke. Growths such as skin tags, warts, brown rough patches (seborrheic keratoses), and other blemishes can be common in older people. In addition, keloids (scars) can arise in older people.

Methods are provided herein to rejuvenate an aged epidermal tissue. In one aspect of the disclosure, a method is provided for rejuvenating aged skin by administering to an individual (e.g., an aged individual, e.g., having an aged skin) or to an aged skin a 15-PGDH inhibitor in an amount effective to inhibit 15-PGDH activity and/or to reduce 15-PGDH levels in the individual, thereby rejuvenating the aged skin. In some cases, the administering is systemic administration (e.g., orally). In other cases, the administering is local administration (e.g., directly to the skin, e.g., by topical administration). In some cases, the administering is topical administration or intradermal injection. Any suitable administration route as described herein may be used.

In various aspects, after administration of the 15-PGDH inhibitor, a function of the aged epidermal tissue is rejuvenated relative to a function of the aged epidermal tissue prior to treatment with the 15-PGDH inhibitor. In various aspects, after administration of the 15-PGDH inhibitor, a function of the aged epidermal tissue is rejuvenated to a level substantially similar to a level found in young epidermal tissue.

In some embodiments, after administration of the 15-PGDH inhibitor, the aged epidermal tissue exhibits enhanced skin condition. In some embodiments, after administration of the 15-PGDH inhibitor, the aged epidermal tissue exhibits increased barrier function. In some embodiments, after administration of the 15-PGDH inhibitor, the aged epidermal tissue supports increased hair growth. The administration of the 15-PGDH inhibitor can counter baldness. The administration of the 15-PGDH inhibitor can stimulate hair follicle stem cells. In some embodiments, after administration of the 15-PGDH inhibitor, the aged epidermal tissue exhibits increased elasticity of skin. The administration of the 15-PGDH inhibitor can counter pattern baldness or alopecia.

### Rejuvenating Aged Brain

The brain is made up of several functional parts, each with a specific and important function. The functional parts can include frontal lobe, temporal lobe, parietal lobe, occipital lobe, cerebellum and brain stem. The brain can control our ability to balance, walk, talk, eat, process information, make decision and feel emotions. The brain can coordinate and regulate breathing, blood circulation, hormone release, and heart rate.

Aging can cause changes to the brain size, vasculature, and cognition. The brain may shrink with increasing age and there may be changes at all levels from molecules to morphology. Incidence of stroke, white matter lesions, dementia and level of memory impairment can also rise with age. There may be changes in levels of neurotransmitters and hormones during aging. Aging may have its effects on the molecules, cells, vasculature, gross morphology, and cognition. In some cases, aging can cause physical changes on the brain. The shrinking of grey matter may due to neuronal cell death. Declining in neuronal volume rather than number may contribute to the changes in an aging brain and that it may be related to sex with different areas most affected in men and women. There may be changes in dendritic arbor, spines, or synapses. In some cases, aging can cause cognitive change on the brain. Cognitive change associated with aging can be that of memory. Memory function can be broadly divided into four sections, episodic memory, semantic memory, procedural memory, and working memory. Episodic memory performance may decline from middle age onwards, and may also be a characteristic of the memory loss seen in Alzheimer's disease (AD). Levels of neurotransmitters (e.g., dopamine and serotonin) may fall as brain ages. Other factors that have been implicated in the aging brain include calcium dysregulation, mitochondrial dysfunction, and the production of reactive oxygen species. Hormone (e.g., sex hormone and growth hormone) levels may also decline as brain ages.

Methods are provided herein to rejuvenate an aged brain. In one aspect of the disclosure, a method is provided for rejuvenating aged brain by administering to an individual (e.g., an aged individual, e.g., having an aged brain) or to an aged brain a 15-PGDH inhibitor in an amount effective to inhibit 15-PGDH activity and/or to reduce 15-PGDH levels in the individual, thereby rejuvenating the aged brain. In some cases, the administering is systemic administration (e.g., orally). In other cases, the administering is local administration (e.g., directly to the brain). In some cases, the administering is intrathecal administration. In some cases, the administering is intracerebroventricular (ICV) administration. Any suitable administration route as described herein may be used.

In various aspects, after administration of the 15-PGDH inhibitor, a function of the aged brain is rejuvenated relative to a function of the aged brain prior to treatment with the 15-PGDH inhibitor. In various aspects, after administration of the 15-PGDH inhibitor, a function of the aged brain is rejuvenated to a level substantially similar to a level found in young brain.

In some embodiments, after administration of the 15-PGDH inhibitor, the aged brain exhibits increased brain size. For example, the aged brain may exhibit increased grey matter, increased amount of neuronal cells, or increased neuronal volume after administration. In some embodiments, after administration of the 15-PGDH inhibitor, the aged brain exhibits improved cognitive performance. For example, the aged brain may exhibit improved memory performance after administration. In some embodiments, after administration of the 15-PGDH inhibitor, the aged brain exhibits increased level of neurotransmitters such as dopamine, serotonin and other brain-derived neurotrophic factors. In some embodiments, after administration of the 15-PGDH inhibitor, the aged brain exhibits increased level of hormones (e.g., oestrogen and growth hormone). In some embodiments, after administration of the 15-PGDH inhibitor, the aged brain exhibits reduced risk of stroke, white matter lesions, or dementia. In some embodiments, after administration of the 15-PGDH inhibitor, the aged brain exhibits reduced risk of Alzheimer's or Parkinson's disease. In some embodiments, after administration of the 15-PGDH inhibitor, the aged brain of a subject having a Alzheimer's or Parkinson's disease may be prevented from worsening. In some embodiments, after administration of the 15-PGDH inhibitor, cognitive performance of the aged brain of a subject having a Alzheimer's or Parkinson's disease may be restored. For example, plaques or tangles associated with Alzheimer's disease may be reduced or may be inhibited from further developing.

Rejuvenating Aged Heart and/or Cardiac Muscle

The cardiac muscle is a specialized type of muscle tissue that forms the heart. This muscle tissue, which contracts and releases involuntarily, is responsible for keeping the heart pumping blood around the body. Cardiac muscle comprises cardiac muscle cells or cardiomyocytes (also known as myocardiocytes or cardiac myocytes are the muscle cells (myocytes) that make up the cardiac muscle (heart muscle). Each cardiac muscle cell contains myofibrils, which are specialized organelles consisting of long chains of sarcomeres, the fundamental contractile units of muscle cells.

Cardiomyocytes show striations similar to those on skeletal muscle cells. Cardiomyocytes have a high mitochondrial density, which allows them to produce adenosine triphosphate (ATP) quickly, making them highly resistant to fatigue. The cardiac muscle's main function includes pumping the blood, which includes plasma, red blood cells, white blood cells, and platelets that is circulated by the heart through the vertebrate vascular system, carrying oxygen and nutrients to and waste materials away from all body tissues. During the aging process, one or more of these functions (e.g., pumping oxygenated blood to the other body parts; pumping hormones and other vital substances to different parts of the body; receiving deoxygenated blood and carrying metabolic waste products from the body and pumping it to the lungs for oxygenation; or maintaining blood pressure) can be abrogated or reduced. In elderly people, cardiac muscle function has been shown to be decreased or impaired. In some cases, the aged cardiac muscle has been shown to exhibit similar impairment as vascular tissue such as oxidative stress, mitochondrial dysfunction, impaired resistance to molecular stressors, chronic low-grade inflammation, genomic instability, cellular senescence, epigenetic alterations, loss of protein homeostasis, deregulated nutrient sensing, or stem cell dysfunction. In some cases, the aged cardiac muscle can impact heart function and anatomy. The heart has a natural pacemaker system that controls the heartbeat. Some of the pathways of this system can develop fibrous tissue and fat deposits due to aging. The natural pacemaker (the sinoatrial or SA node) loses some of its cells. These changes can result in a slightly slower heart rate. Aging can also lead to increase in the size of the heart, especially the left ventricle. The heart wall thickens, so the amount of blood that the chamber can hold may actually decrease despite the increased overall heart size. The heart can fill more slowly. Heart changes often cause the electrocardiogram (ECG) of a normal, healthy older person to be slightly different than the ECG of a healthy younger adult. Abnormal rhythms (arrhythmias), such as atrial fibrillation, are more common in aging cardiac muscle. They may be caused by several types of heart disease. Normal changes in the heart include deposits of the “aging pigment,” lipofuscin. The heart muscle cells degenerate slightly. The valves inside the heart, which control the direction of blood flow, thicken and become stiffer. A heart murmur caused by valve stiffness can be common in aging heart.

Accordingly, in one aspect of the disclosure, a method is provided for rejuvenating aged cardiac muscle by administering to an individual (e.g., an aged individual, e.g., having an aged cardiac muscle) or to an aged cardiac muscle a 15-PGDH inhibitor in an amount effective to inhibit 15-PGDH activity and/or to reduce 15-PGDH levels in the individual, thereby rejuvenating the aged cardiac muscle. In some cases, the administering is systemic administration (e.g., orally or intravenously). In other cases, the administering is local administration (e.g., intraventricularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged cardiac muscle is rejuvenated relative to a function of the aged cardiac muscle prior to treatment with the 15-PGDH inhibitor. In various aspects, after administration of the 15-PGDH inhibitor, a function of the aged cardiac muscle is rejuvenated to a level substantially similar to a level found in young cardiac muscle.

In some embodiments, after administration of the 15-PGDH inhibitor, the aged cardiac muscle exhibits increased or enhanced cardiac muscle tissue functions (e.g., as compared to the aged cardiac muscle prior to treatment with the 15-PGDH inhibitor) as determined by: echocardiogram, transesophageal echocardiography (TEE), electrocardiogram (ECG or EKG), magnetic resonance imaging (MRI), CT scan, exercise cardiac stress test, pharmacologic stress test, tilt test, ambulatory rhythm monitoring tests, or coronary angiogram. In some embodiments, after administration of the 15-PGDH inhibitor, the aged cardiac muscle is capable of functioning at substantially similar to levels found in young cardiac muscle. In some embodiments, after administration of the 15-PGDH inhibitor, the aged cardiac muscle is capable of functioning such as pumping oxygenated blood to the other body parts; pumping hormones and other vital substances to different parts of the body; receiving deoxygenated blood and carrying metabolic waste products from the body and pumping it to the lungs for oxygenation; or maintaining blood pressure at levels that are substantially similar to levels found in young cardiac muscle. In some embodiments, after administration of the 15-PGDH inhibitor, the aged cardiac muscle exhibits decreased fibrosis or exhibits a fibrosis level that is substantially similar to a fibrosis level in young cardiac muscle.

### Rejuvenating Aged Bone

Bone is a rigid tissue that constitutes part of the vertebrate skeleton. Bones protect the various organs of the body, produce red and white blood cells, store minerals, provide structure and support for the body, and enable mobility. Bone tissue (osseous tissue) is a hard tissue, a type of specialized connective tissue and made up of different types of bone cells. Osteoblasts and osteocytes are involved in the formation and mineralization of bone; osteoclasts are involved in the resorption of bone tissue. Modified (flattened) osteoblasts become the lining cells that form a protective layer on the bone surface. The mineralized matrix of bone tissue has an organic component of mainly collagen called ossein and an inorganic component of bone mineral made up of various salts. Other types of tissue found in bones include bone marrow, endosteum, periosteum, nerves, and blood vessels.

The bone's main function includes mechanical support and movement: hematopoiesis; storage of mineral or fat; stabilizing pH or calcium; hormone secretion; lubrication; or damages repair. During the aging process, one or more of these functions may be abrogated or reduced. In elderly people, bone function has been shown to be decreased or impaired. In addition, aged bone can lead to osteomalacia, or osteoporosis.

Accordingly, in one aspect of the disclosure, a method is provided for rejuvenating aged bone by administering to an individual (e.g., an aged individual, e.g., having an aged bone) or to an aged bone a 15-PGDH inhibitor in an amount effective to inhibit 15-PGDH activity and/or to reduce 15-PGDH levels in the individual, thereby rejuvenating the aged bone. In some cases, the administering is systemic administration (e.g., orally or intravenously). In other cases, the administering is local administration (e.g., intraosseous infusion). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged bone is rejuvenated relative to a function of the aged bone prior to treatment with the 15-PGDH inhibitor. In various aspects, after administration of the 15-PGDH inhibitor, a function of the aged bone is rejuvenated to a level substantially similar to a level found in young bone.

In some embodiments, after administration of the 15-PGDH inhibitor, the aged bone exhibits increased or enhanced bone tissue functions (e.g., as compared to the aged bone prior to treatment with the 15-PGDH inhibitor) as determined by: mechanical methods (e.g., whole-bone mechanical testing, bulk tissue specimen mechanical testing, microbeam mechanical testing, microindentation, or nanoindentation); imaging methods (e.g., computerized tomography (CT), magnetic resonance imaging (MRI), nuclear magnetic resonance (NMR), fourier-transform infrared spectroscopy (FTIR), Raman imaging, or scanning electron microscopy); chemical or physical methods (gravimetric analysis or chemical analysis of collagen crosslinks); bone densitometry.

In some embodiments, after administration of the 15-PGDH inhibitor, the aged bone is capable of functioning at substantially similar to levels found in young bone. In some embodiments, after administration of the 15-PGDH inhibitor, the aged bone is capable of functioning such as mechanical support and movement, angiogenesis, storage of mineral or fat, stabilizing pH or calcium, hormone secretion, or lubrication at levels that are substantially similar to levels of function found in young bone. In some embodiments, after administration of the 15-PGDH inhibitor, the aged bone exhibits decreased fibrosis or exhibits a fibrosis level that is substantially similar to a fibrosis level in young bone.

Rejuvenating Aged Sensory Organs (e.g., Organs Involved in Sight, Taste. Hearing, Touch, Smell)

Sensory organs are organs that sense and transduce stimuli and can include eyes, ears, skin, nose, or mouth that correspond to a respective visual system (sense of vision), auditory system (sense of hearing), somatosensory system (sense of touch), olfactory system (sense of smell), and gustatory system (sense of taste). In some cases, the sensory organs are for sensing internal sensation, or interoception, for detecting stimuli from internal organs and tissues. Such sensory organs can include vestibular system (sense of balance) sensed by the inner ear and providing the perception of spatial orientation; proprioception (body position); and nociception (pain). Additionally, sensory organ can include internal chemoreception or and osmoreception-based sensory systems, leading to various perceptions such as hunger, thirst, suffocation, nausea, or different involuntary behaviors, such as vomiting.

The sensory organ's main function includes sensing stimuli such as physical stimuli such as pressure and vibration, sensation of sound, or body position (balance); light (visible electromagnetic radiation); chemical stimuli such as taste or smell; pain; temperature; or other internal stimuli. During the aging process, one or more of these functions may be abrogated or reduced. In elderly people, sensory organ function has been shown to be decreased or impaired.

Accordingly, in one aspect of the disclosure, a method is provided for rejuvenating aged sensory organ by administering to an individual (e.g., an aged individual, e.g., having an aged sensory organ) or to an aged sensory organ a 15-PGDH inhibitor in an amount effective to inhibit 15-PGDH activity and/or to reduce 15-PGDH levels in the individual, thereby rejuvenating the aged sensory organ. In some cases, the administering is systemic administration (e.g., orally or intravenously). In other cases, the administering is local administration (e.g., intraocularly or intranasally). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged sensory organ is rejuvenated relative to a function of the aged sensory organ prior to treatment with the 15-PGDH inhibitor. In various aspects, after administration of the 15-PGDH inhibitor, a function of the aged sensory organ is rejuvenated to a level substantially similar to a level found in young sensory organ.

In some embodiments, after administration of the 15-PGDH inhibitor, the aged sensory organ exhibits increased or enhanced sensory organ tissue functions (e.g., as compared to the aged sensory organ prior to treatment with the 15-PGDH inhibitor) as determined by: visual test, hearing test, olfactory test, physical fitness or balance test, or any other tests that examine internal or external senses or stimuli.

In some embodiments, after administration of the 15-PGDH inhibitor, the aged sensory organ is capable of functioning at substantially similar to levels found in young sensory organ. In some embodiments, after administration of the 15-PGDH inhibitor, the aged sensory organ is capable of functioning such as sensing stimuli such as physical stimuli such as pressure and vibration, sensation of sound, or body position (balance); light (visible electromagnetic radiation); chemical stimuli such as taste or smell; pain; temperature; or other internal stimuli at levels that are substantially similar to levels of function found in young sensory organ. In some embodiments, after administration of the 15-PGDH inhibitor, the aged eye exhibits decreased level of dry eye disease, lacrimal gland inflammation, or macular degeneration.

### Rejuvenating Aged Kidney

Kidney is an organ that receives blood from renal arteries, and the blood exits into renal veins. Kidney is also connected to a ureter, which carries urine to the bladder. Exemplary cell types of kidney include glomerulus parietal cell, glomerulus podocyte, proximal tubule brush border cell, Loop of Henle thin segment cell, thick ascending limb cell, distal tubule cell, collecting duct principal cell, collecting duct intercalated cell, or interstitial kidney cells. The kidney participates in the control of the volume of various body fluids, fluid osmolality, acid-base balance, various electrolyte concentrations, and removal of toxins. Filtration occurs in the glomerulus: one-fifth of the blood volume that enters the kidneys is filtered. Examples of substances reabsorbed are solute-free water, sodium, bicarbonate, glucose, and amino acids. Examples of substances secreted are hydrogen, ammonium, potassium and uric acid. The kidney also carries out functions independent of the nephron. For example, they convert a precursor of vitamin D to its active form, calcitriol; and synthesize the hormones erythropoietin and renin.

The kidney's main function includes formation of urine (e.g., filtration, reabsorption, secretion, or excretion); hormone secretion; blood pressure regulation; acid-base balance; or regulation of osmolality. During the aging process, one or more of these functions may be abrogated or reduced. In elderly people, kidney function has been shown to be decreased or impaired.

Accordingly, in one aspect of the disclosure, a method is provided for rejuvenating aged kidney by administering to an individual (e.g., an aged individual, e.g., having an aged kidney) or to an aged kidney a 15-PGDH inhibitor in an amount effective to inhibit 15-PGDH activity and/or to reduce 15-PGDH levels in the individual, thereby rejuvenating the aged kidney. In some cases, the administering is systemic administration (e.g., orally). In other cases, the administering is local administration (e.g., intravenously or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged kidney is rejuvenated relative to a function of the aged kidney prior to treatment with the 15-PGDH inhibitor. In various aspects, after administration of the 15-PGDH inhibitor, a function of the aged kidney is rejuvenated to a level substantially similar to a level found in young kidney.

In some embodiments, after administration of the 15-PGDH inhibitor, the aged kidney exhibits increased or enhanced kidney tissue functions (e.g., as compared to the aged kidney prior to treatment with the 15-PGDH inhibitor) as determined by: clinical assessment; urine tests; blood tests (e.g., glomerular filtration rate); medical imaging (e.g., CT scan); or biopsy.

In some embodiments, after administration of the 15-PGDH inhibitor, the aged kidney is capable of functioning at substantially similar to levels found in young kidney. In some embodiments, after administration of the 15-PGDH inhibitor, the aged kidney is capable of functioning such as formation of urine (e.g., filtration, reabsorption, secretion, or excretion); hormone secretion; blood pressure regulation; acid-base balance; or regulation of osmolality at levels that are substantially similar to levels of function found in young kidney. In some embodiments, after administration of the 15-PGDH inhibitor, the aged kidney exhibits decreased level of kidney disease such as chronic kidney disease, nephritic and nephrotic syndromes, acute kidney injury, pyelonephritis, or kidney cancer. In some embodiments, after administration, the kidney is protected from ischemic renal injury, exhibits increased vasodilation, exhibits increased renal blood flow, exhibits reduced biomarkers of renal injury, exhibits induction of PGE2 levels, and/or exhibits induction of PGE2 receptors.

### Rejuvenating Aged Thyroid

Thyroid is an endocrine gland that comprises spherical thyroid follicle, lined with follicular cells (thyrocytes), and parafollicular cells that surround a lumen containing colloid. The thyroid gland secretes three hormones: the two thyroid hormones: triiodothyronine (T3) and thyroxine (T4); and a peptide hormone, calcitonin. The thyroid hormones influence the metabolic rate and protein synthesis. Calcitonin plays a role in calcium homeostasis. The thyroid's main function includes regulating, producing, and secreting hormones. During the aging process, one or more of these functions may be abrogated or reduced. In elderly people, thyroid function has been shown to be decreased or impaired.

Accordingly, in one aspect of the disclosure, a method is provided for rejuvenating aged thyroid by administering to an individual (e.g., an aged individual, e.g., having an aged thyroid) or to an aged thyroid a 15-PGDH inhibitor in an amount effective to inhibit 15-PGDH activity and/or to reduce 15-PGDH levels in the individual, thereby rejuvenating the aged thyroid. In some cases, the administering is systemic administration (e.g., orally). In other cases, the administering is local administration (e.g., intravenously or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged thyroid is rejuvenated relative to a function of the aged thyroid prior to treatment with the 15-PGDH inhibitor. In various aspects, after administration of the 15-PGDH inhibitor, a function of the aged thyroid is rejuvenated to a level substantially similar to a level found in young thyroid.

In some embodiments, after administration of the 15-PGDH inhibitor, the aged thyroid exhibits increased or enhanced thyroid tissue functions (e.g., as compared to the aged thyroid prior to treatment with the 15-PGDH inhibitor) as determined by: blood tests for measuring thyroid-stimulating hormone (TSH), triiodothyronine (T3), thyroxine (T4), or calcitonin; antibody tests for detecting thyroid hormones; or radioactive iodine uptake.

In some embodiments, after administration of the 15-PGDH inhibitor, the aged thyroid is capable of functioning at substantially similar to levels found in young thyroid. In some embodiments, after administration of the 15-PGDH inhibitor, the aged thyroid is capable of functioning such as regulating, producing, and secreting hormones at levels that are substantially similar to levels of function found in young thyroid. In some embodiments, after administration of the 15-PGDH inhibitor, the aged thyroid exhibits decreased level of thyroid disease such as hyperthyroidism, hypothyroidism, Hashimoto's thyroiditis, Graves' disease, goiter, thyroid nodule, or thyroid cancer.

### Rejuvenating Lung

Lung is an organ of the respiratory system and comprises various cell types in lung connective tissue, respiratory epithelium, bronchial airways, respiratory zone, or alveoli. Example cell types of lung include alveolar epitheliums. The lung's main function includes gas exchange between lung and blood; protection against respiratory pathogen or infection; maintaining homeostasis of pressure or acid-base in blood; or speech by providing air and airflow for the creation of vocal sound. During the aging process, one or more of these functions may be abrogated or reduced. In elderly people, lung function has been shown to be decreased or impaired.

Accordingly, in one aspect of the disclosure, a method is provided for rejuvenating aged lung by administering to an individual (e.g., an aged individual, e.g., having an aged lung) or to an aged lung a 15-PGDH inhibitor in an amount effective to inhibit 15-PGDH activity and/or to reduce 15-PGDH levels in the individual, thereby rejuvenating the aged lung. In some cases, the administering is systemic administration (e.g., orally). In other cases, the administering is local administration (e.g., by inhalation or intranasally). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged lung is rejuvenated relative to a function of the aged lung prior to treatment with the 15-PGDH inhibitor. In various aspects, after administration of the 15-PGDH inhibitor, a function of the aged lung is rejuvenated to a level substantially similar to a level found in young lung.

In some embodiments, after administration of the 15-PGDH inhibitor, the aged lung exhibits increased or enhanced lung tissue functions (e.g., as compared to the aged lung prior to treatment with the 15-PGDH inhibitor) as determined by: evaluating lung capacity for volume or air inhaled or exhaled; pulmonary plethysmographs, spirometry; lung diffusing capacity; pulse oximetry; lung imaging; bronchoscopy; or thoracotomy.

In some embodiments, after administration of the 15-PGDH inhibitor, the aged lung is capable of functioning at substantially similar to levels found in young lung. In some embodiments, after administration of the 15-PGDH inhibitor, the aged lung exhibits decreased level of lung disease such as inflammation, infection, blood-supply change, obstructive lung disease, restrictive lung disease, congenital disorder, pneumothorax, lung nodule, or lung cancer. In some embodiments, after administration of the 15-PGDH inhibitor, the aged lung exhibits decreased level of lung fibrosis.

### Rejuvenating Aged Smooth Muscle

Smooth muscle is an involuntary non-striated muscle. It is divided into two subgroups; single-unit (unitary) and multiunit smooth muscle. Within single-unit cells, the whole bundle or sheet contracts as a syncytium. Smooth muscle cells (myocytes) are found in the walls of hollow organs, including the stomach, intestines, urinary bladder and uterus, and in the walls of passageways, such as the arteries and veins of the circulatory system, and the tracts of the respiratory, urinary, and reproductive systems. Smooth muscles' main function includes contraction and relaxation, which lead to movement of the digestive tract, movement of the autonomous nervous system (e.g., for breathing), or regulating homeostasis such as raising skin hair follicles for regulating body temperature. During the aging process, one or more of these functions may be abrogated or reduced. In elderly people, smooth muscle function has been shown to be decreased or impaired.

Accordingly, in one aspect of the disclosure, a method is provided for rejuvenating aged smooth muscle by administering to an individual (e.g., an aged individual, e.g., having an aged smooth muscle) or to an aged smooth muscle a 15-PGDH inhibitor in an amount effective to inhibit 15-PGDH activity and/or to reduce 15-PGDH levels in the individual, thereby rejuvenating the aged smooth muscle. In some cases, the administering is systemic administration (e.g., orally). In other cases, the administering is local administration (e.g., by intramuscularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged smooth muscle is rejuvenated relative to a function of the aged smooth muscle prior to treatment with the 15-PGDH inhibitor. In various aspects, after administration of the 15-PGDH inhibitor, a function of the aged smooth muscle is rejuvenated to a level substantially similar to a level found in young smooth muscle.

In some embodiments, after administration of the 15-PGDH inhibitor, the aged smooth muscle exhibits increased or enhanced smooth muscle functions (e.g., as compared to the aged smooth muscle prior to treatment with the 15-PGDH inhibitor) as determined by: blood test for measuring antibody associated with smooth muscle or medical imaging.

In some embodiments, after administration of the 15-PGDH inhibitor, the aged smooth muscle is capable of functioning at substantially similar to levels found in young smooth muscle. In some embodiments, after administration of the 15-PGDH inhibitor, the aged smooth muscle exhibits decreased level of smooth muscle disease such as multisystemic smooth muscle dysfunction syndrome, blood vessel disorders, atherosclerosis, or inflammation. In some embodiments, after administration of the 15-PGDH inhibitor, the aged smooth muscle exhibits decreased level of smooth muscle fibrosis. In some embodiments, rejuvenation of smooth muscle may cause rejuvenation of stomach, digestive tract, urinary tract and/or uterus function. In some embodiments, rejuvenation of smooth muscle may improve iris function in the eye, improve skin function, and sensitivity to temperature changes or adrenalin changes.

### Rejuvenating Aged Blood

Blood is a body fluid that delivers necessary substances such as nutrients and oxygen to the cells and transports metabolic waste products away from those same cells and comprises blood plasma (about 55% of blood fluid,) comprising proteins, glucose, mineral ions, hormones, carbon dioxide (plasma being the main medium for excretory product transportation), and blood cells, mainly red blood cells (RBCs or erythrocytes), white blood cells (WBCs or leukocytes), and platelets. Blood is circulated around the body through blood vessels by the pumping action of the heart., Arterial blood carries oxygen from inhaled air to the tissues of the body, and venous blood carries carbon dioxide, a waste product of metabolism produced by cells, from the tissues to the lungs to be exhaled. Blood's main function includes supply of oxygen to tissues (bound to hemoglobin in red blood cells), supply of nutrients such as glucose, amino acids, or fatty acids (dissolved in the blood or bound to plasma proteins); removal of waste such as carbon dioxide, urea, or lactic acid; immune response, including circulation of white blood cells and detection of foreign material by antibodies; coagulation, messenger function, including transport of hormones; or regulation of core body temperature. During the aging process, one or more of these functions may be abrogated or reduced. In elderly people, blood function has been shown to be decreased or impaired.

Accordingly, in one aspect of the disclosure, a method is provided for rejuvenating aged blood by administering to an individual (e.g., an aged individual, e.g., having an aged blood) or to an aged blood a 15-PGDH inhibitor in an amount effective to inhibit 15-PGDH activity and/or to reduce 15-PGDH levels in the individual, thereby rejuvenating the aged blood. In some cases, the administering is systemic administration (e.g., orally). In other cases, the administering is local administration (e.g., by intravenously or intraventricularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor, a function of the aged blood is rejuvenated relative to a function of the aged blood prior to treatment with the 15-PGDH inhibitor. In various aspects, after administration of the 15-PGDH inhibitor, a function of the aged blood is rejuvenated to a level substantially similar to a level found in young blood. In some embodiments, after administration of the 15-PGDH inhibitor, the aged blood exhibits restored or rejuvenated serum cytokine levels that are substantially similar to serum cytokine levels found in a young individual. The restored or rejuvenated serum cytokine levels in the treated aged blood can be any one or any combination of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1b), interleukin-22 (IL22).

In some embodiments, after administration of the 15-PGDH inhibitor, the aged blood exhibits increased or enhanced blood functions (e.g., as compared to the aged blood prior to treatment with the 15-PGDH inhibitor) as determined by: complete blood count; metabolic panel for measuring metabolites such as electrolytes, calcium, glucose, sodium, potassium, carbon dioxide, chloride, blood urea nitrogen (BUN), creatinine, albumin, total protein, alkaline phosphatase, alkaline aminotransferase, aspartate aminotransferase, or bilirubin; lipid panel; thyroid panel; enzyme markers; coagulation panel; dehydroepiandrosterone (DHEA)-sulfate serum test; C-reactive protein test; or circulating cytokines (e.g., by detecting and measuring serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin−1 beta (IL1b), interleukin-22 (IL22), or any combination thereof.)

In some embodiments, after administration of the 15-PGDH inhibitor, the aged blood is capable of functioning at substantially similar to levels found in young blood. In some embodiments, after administration of the 15-PGDH inhibitor, the aged blood is capable of functioning such as supply of oxygen to tissues (bound to hemoglobin in red blood cells); supply of nutrients such as glucose, amino acids, or fatty acids (dissolved in the blood or bound to plasma proteins); removal of waste such as carbon dioxide, urea, or lactic acid; immune response, including circulation of white blood cells and detection of foreign material by antibodies; coagulation, messenger function, including transport of hormones; or regulation of core body temperature at levels that are substantially similar to levels of function found in young blood. In some embodiments, after administration of the 15-PGDH inhibitor, the aged blood exhibits decreased level of blood disease such as anemia, hemophilia, blood clots, and blood cancers such as leukemia, lymphoma, or myeloma.

### Subjects

The subject can be any subject, e.g., a human or other mammal, with an age-related condition or at risk of having an age-related condition. In some embodiments, the subject is a human. In some embodiments, the subject is an adult. In some embodiments, the subject is a child (e.g., a child with a genetic disorder that causes premature aging, e.g., progeria). In some embodiments, the subject is female (e.g., an adult female). In some embodiments, the subject is male (e.g., an adult male).

In some embodiments, the subject is human, and the method further comprises a step in which the human is selected for treatment with the 15-PGDH inhibitor based on a diagnosis of an age-related condition or disease, or on the potential for or risk of developing an age-related condition or disease, or based on the age of the individual, or based on the presence of one or more biomarkers of aging as described herein. In some such embodiments, the human is selected based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has an age-related condition or disease or has one or more biomarkers of aging as described herein. In some embodiments, the human is selected based on a potential for an age-related condition or disease, based on the presence or potential presence of an environmental, lifestyle, or medical factor linked to premature aging of one or more non-skeletal muscle tissues and/or organs, such as smoking, drinking, diet, lack of physical activity, insufficient sleep, drug use, exposure to UV rays, exposure to extreme temperatures, stress, excess weight, or health-related factors such as infections, mental illness, cancer, diabetes, etc. In some embodiments, the subject has an age-related condition caused by premature aging of one or more tissues, e.g., a genetic disorder such as Osteogenesis imperfecta. Bloom syndrome, Cockayne Syndrome, Hutchinson-Gilford Progeria Syndrome, Mandibuloacral Dysplasia. Progeria, Progeroid Syndrome, Rothmund-Thomson Syndrome, Seip Syndrome, Werner Syndrome, Down Syndrome, Acrogeria, Rothmund-Thomson syndrome, an immunodeficiency of these tissues that lead to premature aging syndromes, such as Ataxia telangiectasia, or an infectious disease of these tissues that lead to premature aging syndromes, such as human immunodeficiency virus (HIV).

In some embodiments, the subject is determined to have aged tissues and/or organs or have an age-related condition or disease as determined using any method of assessing any measure of the function, performance, health, strength, endurance, physiological activity, or any other property of a non-skeletal muscle tissue and/or organ, e.g., a performance-based, imaging-based, physiological, molecular, cellular, or functional assay. For example, a heart can be assessed using any method of assessing heart function or health, such as angiograms, electrocardiograms, treadmill test, echocardiogram, etc. In some embodiments, the subject is selected for treatment based on a detection of elevated levels of 15-PGDH transcript, protein, or enzymatic activity in a non-skeletal muscle related tissue and/or organ, or on a detection of decreased levels of PGE2 and/or PGD2 in the tissue and/or organ.

In some embodiments, the methods comprise an additional step subsequent to the administration of a 15-PGDH inhibitor, comprising assessing the health, function, performance, or any other property of a non-skeletal muscle tissue and/or organ in the subject, or comprising assessing the level of 15-PGDH (e.g., of 15-PGDH protein, transcript, or activity) and/or PGE2 and/or PGD2 in the non-skeletal muscle tissue and/or organ in the subject, e.g., to ascertain the potential effects of the prior administration of the 15-PGDH inhibitor on the tissue and/or organ. In some such embodiments, the health, function, performance, 15-PGDH level, 15-PGDH activity, PGE2 level, PGD2 level, or other property of the tissue and/or organ is detected or examined and compared to the health, function, performance, 15-PGDH level, 15-PGDH activity. PGE2 level, PGD2 level, or other property of the tissue and/or organ prior to the administration of the 15-PGDH inhibitor or to a control value, wherein a determination that the health, function, or performance of the tissue and/or organ has improved, that the 15-PGDH level has decreased, that the 15-PGDH activity has decreased, that the PGE2 level and/or PGD2 level has increased, in the tissue subsequent to the administration of the 15-PGDH inhibitor as compared to the value obtained prior to the administration of the 15-PGDH inhibitor or relative to a control value, indicates that the 15-PGDH inhibitor has had a beneficial effect in the non-skeletal muscle tissue and/or organ of the subject.

In some embodiments, the subject has an age-related condition, disorder, or disease such as a cardiovascular disease or condition (e.g., atrial fibrillation, stroke, ischemic heart diseases, cardiomyopathies, endocarditis, intracerebral haemorrhage, hypertension), a chronic respiratory disease or condition (e.g., chronic obstructive pulmonary disease, asbestosis, silicosis), a nutritional disease or condition (e.g., trachoma, diarrheal diseases, encephalitis), a kidney disease or condition (e.g., chronic kidney diseases), a gastrointestinal or digestive disease or condition (e.g., NASH, pancreatitis, ulcer, intestinal obstruction), a neurological disorder (e.g., Alzheimer's, dementia, Parkinson's, cognitive decline), a sensory disorder (e.g., hearing loss, vision loss, loss of sense of smell or sense of taste, macular degeneration, retinitis pigmentosa, glaucoma), a skin or subcutaneous disease or condition (e.g., cellulitis, ulcer, fungal skin diseases, pyoderma), osteoporosis, osteoarthritis, rheumatoid arthritis, a genetic disease causing premature aging in one or more non-skeletal muscle tissues (e.g., progeria, osteogenesis imperfecta, Bloom syndrome, Cockayne Syndrome, Hutchinson-Gilford Progeria Syndrome, Mandibuloacral Dysplasia. Progeroid Syndrome, Rothmund-Thomson Syndrome, Seip Syndrome, Werner Syndrome, Down Syndrome, Acrogeria, Rothmund-Thomson syndrome), an immunodeficiency of these tissues that lead to premature aging syndromes (e.g., Ataxia telangiectasia), or an infectious disease of these tissues that leads to premature aging syndromes, (e.g., human immunodeficiency virus (HIV)), and the like.

The administration of the 15-PGDH inhibitor can provide improvement in any of these conditions, and can help improve, e.g., osteoporosis, hair loss, aged skin, cognitive disorders, sensory disorders, aged hematopoietic stem cell function, and gastrointestinal function, as described herein.

The present methods and compositions can be used to treat any non-skeletal muscle tissue, or organs including such tissues, or cells within such tissues, including epithelial tissue, nerve tissue, connective tissue, smooth muscle, cardiac muscle, epidermal tissues, vascular tissues, heart, kidney, brain, bone, cartilage, brown fat, spleen, liver, colon, sensory organs, thyroid, lung, blood, small intestine, dental tissue, ovaries or other reproductive tissue, hair, cochlea, oligodendrocytes, etc.

In some embodiments, subjects are identified for treatment based on a diagnosis of an age-related condition, disorder, or disease; based on a determination of the presence of or potential for age-related loss of non-skeletal muscle tissue and/or organ function, health, or performance; based on a subject's age, e.g., an age associated with an age-related condition or disease; or based on a detection of any of the herein-described features of aged non-skeletal muscle tissues and/or organs, e.g., of elevated levels of PGE2 and/or PGD2 metabolites such as 15-keto-PGE2, PGEM, 15-keto-PGD2, or 13,14-Dihydro-15-PGD2, of decreased levels of PGE2 and/or PGD2, of decreased protein synthesis, of decreased mitochondrial activity, of decreased signaling through the EP1, EP2, EP3, EP4, DP1, and/or DP2 receptors, of elevated expression of genes associated with the senescence phenotype such as p16 (Ink4a) or p21 (Cdkn1a), of shortened telomere length in cells of the tissue, of elevated numbers of senescent cells in a non-skeletal muscle tissue, or of elevated levels or activity of 15-PGDH, in particular in senescent cells, e.g., macrophages, fibroadipocytes, fibroblasts, endothelial cells, etc.

In some embodiments, the subject is a pet or a farm animal such as a porcine, bovine, ovine, poultry, or fish, and the methods are used, e.g., to enhance non-skeletal muscle tissue and/or organ function or health in an aging animal. In some such embodiments, the animal is administered a small molecule inhibitor of 15-PGDH. In some embodiments, a vector or expression cassette comprising a nucleic acid inhibitor of 15-PGDH, e.g., an shRNA, is introduced into the animal such that the nucleic acid inhibitor is expressed in the cells of the animal, e.g., the cells of the non-skeletal muscle tissue and/or organ. In some embodiments, a vector or expression cassette comprising a polynucleotide encoding a polypeptide inhibitor of 15-PGDH, e.g., an antibody or peptide, is introduced into the animal such that the polypeptide inhibitor is expressed in the cells of the animal, e.g., the cells of the non-skeletal muscle tissue and/or organ. In some embodiments, gene therapy is used, e.g., such that all or part of an endogenous 15-PGDH encoding gene is replaced with a form of the gene that is less active, less stable, or less highly expressed in cells, e.g., non-skeletal muscle tissue and/or organ cells, of the animal. In some embodiments, modified RNA, e.g., a chemically modified RNA inhibitor such as shRNA or a chemically modified mRNA encoding a polypeptide 15-PGDH inhibitor is introduced into the animal such that the RNA inhibitor or expressed protein inhibitor is present in cells of the animal.

### 5. Assessing 15-PGDH Levels

Any of a number of methods can be used to assess the level of 15-PGDH in a non-skeletal muscle tissue and/or organ, e.g., when using 15-PGDH as a biomarker or when assessing the efficacy of an inhibitor of 15-PGDH. For example, the level of 15-PGDH can be assessed by examining the transcription of a gene encoding 15-PGDH (e.g., the Hpgd gene), by examining the levels of 15-PGDH protein in the tissue and/or organ (e.g., non-skeletal muscle tissue and/or organ), or by measuring the 15-PGDH enzyme activity in the tissue and/or organ (e.g., non-skeletal muscle tissue and/or organ). Such methods can be performed on the overall tissue and/or organ or on a subset of cells within the tissue and/or organ, e.g., senescent cells.

In some embodiments, the methods involve the measurement of 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

In some embodiments, the methods involve the detection of 15-PGDH-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry. 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of 15-PGDH protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation.” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao. J. Chromatogr. B. Biomed Sci., 699:463-80 (1997)).

### 6. 15-PGDH as a Biomarker

In some embodiments, 15-PGDH may be used as a biomarker for aged non-skeletal muscle tissue and/or organs, or for the presence or potential for an age-related condition or disease. For example, a detection of an increase in 15-PGDH levels in a non-skeletal muscle tissue and/or organ, e.g., in the overall tissue and/or organ or in specific cells within the tissue and/or organ such as senescent cells, is indicative of aging in the tissue and/or organ, of a loss or decrease of function or health of the tissue and/or organ related to aging, or of the presence of an age-related condition or disease. For example, a detected increase of about 10%, about 20%, about 30%, about 40%, about 50%, about 60%, about 70%, about 80%, about 90%, about 100%, or more 15-PGDH in a non-skeletal muscle tissue and/or organ as compared to in a control tissue and/or organ from a subject without an age-related condition or disease may be indicative of aging of the tissue and/or organ, of a loss or decrease of function or health of the tissue and/or organ related to aging, or of the presence of an age-related condition or disease.

### 7. 15-PGDH Inhibitors

Any agent that reduces, decreases, counteracts, attenuates, inhibits, blocks, downregulates, or eliminates in any way the expression, stability, or activity. e.g., enzymatic activity, of 15-PGDH can be used in the present methods. Inhibitors can be small molecule compounds, peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH.

In some embodiments, the 15-PGDH inhibitor decreases the activity, stability, or expression of 15-PGDH by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more relative to a control level, e.g., in the absence of the inhibitor, in vivo or in vitro.

The efficacy of inhibitors can be assessed, e.g., by measuring 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology. 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See. e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the 15-PGDH inhibitor is considered effective if the level of expression of a 15-PGDH-encoding polynucleotide is decreased by at least about 10%, at least about 20%, at least about 30%, at least about 40%, at least about 50%, at least about 60%, at least about 70%, at least about 80%, at least about 90% or more as compared to the reference value. e.g., the value in the absence of the inhibitor, in vitro or in vivo. In some embodiments, a 15-PGDH inhibitor is considered effective if the level of expression of a 15-PGDH-encoding polynucleotide is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, or more as compared to the reference value.

The effectiveness of a 15-PGDH inhibitor can also be assessed by detecting protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation.” Principles of Proteomics, 2nd Edition. R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

For determining whether 15-PGDH protein levels are decreased in the presence of a 15-PGDH inhibitor, the method comprises comparing the level of the protein (e.g., 15-PGDH protein) in the presence of the 15-PGDH inhibitor to a reference value, e.g., the level in the absence of the 15-PGDH inhibitor. In some embodiments, a 15-PGDH protein is decreased in the presence of an inhibitor if the level of the 15-PGDH protein is decreased by at least about 10%, at least about 20%, at least about 30%, at least about 40%, at least about 50%, at least about 60%, at least about 70%, at least about 80%, at least about 90%, or more as compared to the reference value. In some embodiments, a 15-PGDH protein is decreased in the presence of an inhibitor if the level of the 15-PGDH protein is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, or more as compared to the reference value.

### Small Molecules

In particular embodiments, 15-PGDH is inhibited by the administration of a small molecule inhibitor. Any small molecule inhibitor can be used that reduces, e.g., by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more, the expression, stability, or activity of 15-PGDH relative to a control, e.g., the expression, stability, or activity in the absence of the 15-PGDH inhibitor. In particular embodiments, small molecule inhibitors may be used that can reduce the enzymatic activity of 15-PGDH in vitro or in vivo. Non-limiting examples of small molecule compounds that can be used in the present methods include the small molecules disclosed in publication EP 2838533, the entire disclosure of which is herein incorporated by reference. Small molecules can include, inter alia, the small molecules disclosed in Table 2 of publication EP 2838533, i.e., SW033291, SW033291 isomer B, SW033291 isomer A, SW033292, 413423, 980653, 405320, SW208078, SW208079, SW033290, SW208080, SW208081, SW206976, SW206977, SW206978, SW206979, SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4-(2-cyclohexylethoxy)benzylidene)thiazolidine-2,4-dione, 5-(3-chloro-4-(2-cyclohexylethoxy)benzyl)thiazolidine-2,4-dione, (Z)—N-benzyl-4-((2,4-dioxothiazolidin-5-ylidene)methyl)benzamide, or any of the compounds disclosed in Choi et al. (2013) Bioorganic & Medicinal Chemistry 21:4477-4484; Wu et al. (2010) Bioorg. Med. Chem. 18(2010) 1428-1433; Wu et al. (2011) J. Med. Chem. 54:5260-5264; or Yu et al. (2019) Biotechnology and Bioprocess Engineering 24:464-475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins, Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor is 2-((6-bromo-4H-imidazo[4,5-b]pyridin-2-ylthio)methyl)benzonitrile (CID: 3245059), piperidin-1-yl(1-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular Libraries Program [Internet]), the entire disclosure of which is herein incorporated by reference.

In some embodiments, the 15-PGDH inhibitor is TD88 or any of the compounds disclosed in Seo et al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor is EEAH (Ethanol extract of Artocarpus heterophyllus) or any of the compounds disclosed in Kama (2017) Pharmacogn Mag. 2017 January; 13(Suppl 1): S122-S126, the entire disclosure of which is herein incorporated by reference.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical) to a target sequence in a 15-PGDH polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos, NM_000860.6, NM_001145816.2, NM_001256301.1, NM_001256305.1, NM_001256306.1, NM_001256307.1, or NM_001363574.1).

In some embodiments, the methods described herein comprise treating a subject, e.g., a subject with an age-related condition, disorder, or disease, using an shRNA or siRNA. A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject, e.g., a subject with an age-related condition, disorder, or disease, comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos, NM_000860.6, NM_001145816.2, NM_001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression, miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM_000860.6, NM_001145816.2, NM_001256301.1, NM_001256305.1, NM_001256306.1, NM_001256307.1, or NM_001363574.1). In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., about 95%, about 90%, about 85%, about 80%, about 75%, or about 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies, 4^{th} edition by N. S. Templeton; Translating Gene Therapy to the Clinic: Techniques and Approaches. 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see, e.g., Li et al., Nuc. Acids Res. (2006). 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function. e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See, e.g., Kohler & Milstein, Nature 256:495-497 (1975): Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379: WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector. e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see. e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively. Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet™ (FortéBio, Inc., Menlo Park, Calif.)), and western blot analysis.

### Peptides

In some embodiments, the agent is a peptide, e.g., a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, (Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and Modified RNA

In some embodiments, polynucleotides providing 15-PGDH inhibiting activity, e.g., a nucleic acid inhibitor such as an siRNA or shRNA, or a polynucleotide encoding a polypeptide that inhibits 15-PGDH, are introduced into cells, e.g., non-skeletal muscle tissue and/or organ cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitors, e.g., a nucleic acid inhibitor or a polynucleotide encoding a polypeptide inhibitor, are introduced into cells, e.g., non-skeletal muscle tissue and/or organ cells, using vectors such as viral vectors. Suitable viral vectors include but not limited to adeno-associated viruses (AAVs), adenoviruses, and lentiviruses. In some embodiments, a 15-PGDH inhibitor. e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the non-skeletal muscle tissues and/or organs of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection. e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, poly amidoamine, and polyethyleneimine, chitosan, and poly(1-amino esters) can also be used. See, e.g., Youn et al. (2015) Expert Opin Biol Ther, September 2: 15(9): 1337-1348; Kaczmarek et al. (2017) Genome Medicine 9:60; Gan et al. (2019) Nature comm. 10: 871; Chien et al. (2015) Cold Spring Harb Perspect Med. 2015; 5:a014035; the entire disclosures of each of which are herein incorporated by reference.

### 8. Methods of Administration

The compounds described herein can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days. 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

Administration of the compound into a subject can be accomplished by methods generally used in the art. The quantity of the compound introduced may take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount”, “pharmacologically effective dose”, “therapeutically effective dose”, or “therapeutically effective amount” is an amount sufficient to produce the desired physiological effect or amount capable of achieving the desired result, particularly for treating the condition or disease, including reducing or eliminating one or more symptoms or manifestations of the condition or disease.

The compounds described herein may be administered locally by injection into the non-skeletal muscle tissue and/or organ being targeted, or by administration in proximity to the tissue being targeted. The compounds described herein may be administered systemically (e.g., orally) such that multiple tissues and/or organs are treated and/or affected.

### 9. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see, e.g., REMINGTON's PHARMACEUTICAL SCIENCES, 18TH ED., Mack Publishing Co., Easton, Pa. (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g. an ampoule). In addition, more concentrated dosage forms may be prepared, from which the more dilute unit dosage forms may then be produced. The more concentrated dosage forms thus will contain substantially more than, e.g., at least 1, at least 2, at least 3, at least 4, at least 5, at least 6, at least 7, at least 8, at least 9, at least 10, or more times the amount of the therapeutic compound.

Methods for preparing such dosage forms are known to those skilled in the art (see. e.g., REMINGTON's PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON's PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (e.g., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side effects caused by induction of the immune response, etc. In some embodiments, for example, the 15-PGDH inhibitors described herein are administered together with a senolytic agent, a compound to enhance PGE2 levels or PGD2 levels, a compound to decrease Atrogin1 levels or activity, a compound to increase signaling through the EP1, EP2, EP3, EP4, DP1, and/or DP2 receptors, and/or any other compound aiming to enhance muscle mass, strength, or function; or the function, health, or any other desired property of the non-skeletal muscle tissue and/or organ being targeted.

### 10. Kits

Other embodiments of the compositions described herein are kits comprising a 15-PGDH inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of aged non-skeletal muscle tissue and/or organs. In some embodiments, the kit comprises one or more reagents for the treatment of a non-skeletal muscle tissue and/or organs in a subject with an age-related condition, disorder, or disease. In some embodiments, the kit comprises an agent that antagonizes the expression or activity of 15-PGDH. In some embodiments, the kit comprises an inhibitory nucleic acid (e.g., an antisense RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises an antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor, e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH.

In some embodiments, the kits can further comprise instructional materials containing directions (e.g., protocols) for the practice of the methods described herein (e.g., instructions for using the kit for enhancing the function, health, or other properties of non-skeletal muscle tissues and/or organs). While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail by way of a specific example. The following example is offered for illustrative purposes only, and is not intended to limit the disclosure in any manner. Those of skill in the art will readily recognize a variety of noncritical parameters which can be changed or modified to yield essentially the same results.

### Example 1. Targeting Prostaglandin E2 Degrading Enzyme to Ameliorate Non-Skeletal Muscle Tissue Function in Age-Related Diseases and Conditions

As we age, quality of life is reduced and mortality is increased. Age-related diseases are a group of diseases that occur more frequently in people as they age which directly correlate to decreased longevity (l). These age-related diseases include cardiovascular diseases (atrial fibrillation, stroke, ischemic heart diseases, cardiomyopathies, endocarditis, intracerebral hemorrhage), chronic respiratory diseases (chronic obstructive pulmonary disease, asbestosis, silicosis), nutritional diseases (trachoma, diarrheal diseases, encephalitis), kidney diseases (chronic kidney diseases), gastrointestinal and digestive diseases (NASH, pancreatitis, ulcer, intestinal obstruction), neurological disorders (Alzheimer's, dementia, Parkinson's), sensor disorders (hearing loss, macular degeneration, glaucoma), skin and subcutaneous diseases (cellulitis, ulcer, fungal skin diseases, pyoderma), osteoporosis, osteoarthritis, rheumatoid arthritis and the like (2).

We determined previously that PGE2 stimulates muscle stem cells (MuSCs) to regenerate damaged muscles in young mice (3), in good agreement with findings regarding its function in regeneration in other tissues, including bone, colon, liver, and blood (4-6). We reasoned that PGE2 signaling might go awry in aging. Here we demonstrate a previously unrecognized role for the PGE2 degrading enzyme. 15-hydroxyprostaglandin dehydrogenase (15-PGDH), in aged tissues. Partial inhibition of 15-PGDH restores PGE2 and/or PGD2 to youthful levels, and can thereby rejuvenate tissue function. Our findings provide fresh insights into aging and uncover an innovative treatment strategy.

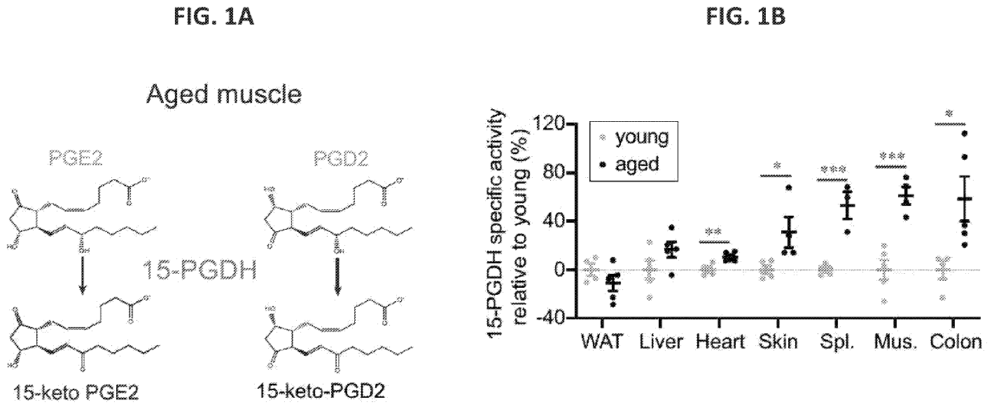

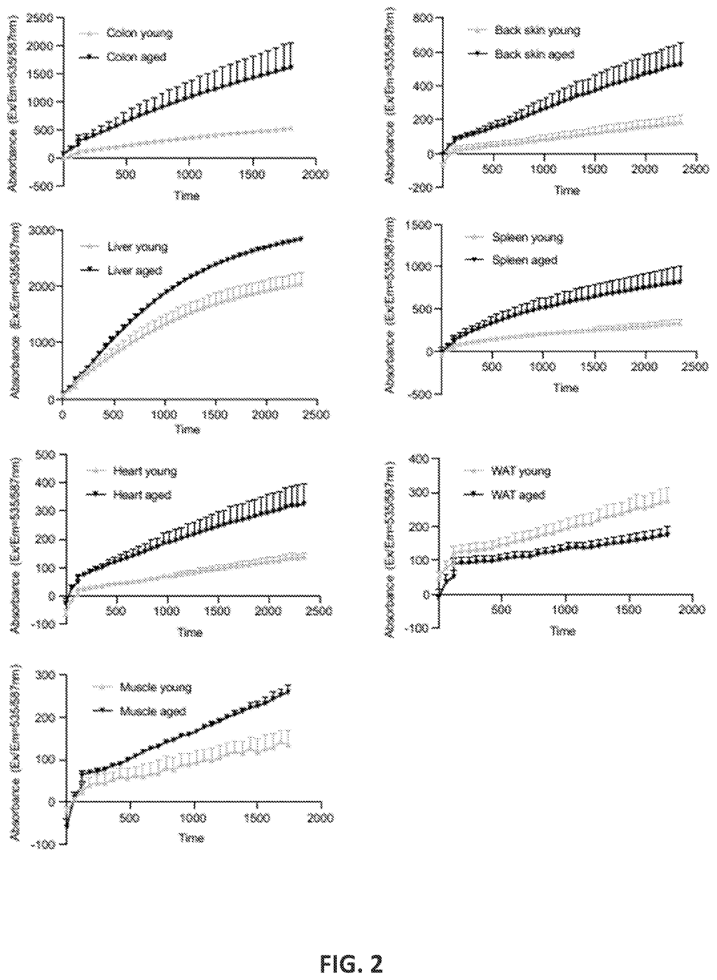

We hypothesized that a reduction in PGE2 was due to increased degradation by 15-PGDH in aged tissues (FIG. 1A). We found that the specific activity of the enzyme was indeed increased in aged tissues, including cardiac, skin, spleen and colon (FIGS. 1B and 2). Accordingly, inhibition of 15-PGDH can help ameliorate age-related diseases and conditions by restoring or increasing PGE2 and/or PGD2 levels in aged tissues.

We uncover 15-PGDH as a new marker of aging, detectable at elevated activity in numerous tissues such as heart, skin, colon, and spleen. Restoring PGE2 and/or PGD2 to youthful levels can therefore provide pleiotropic ameliorative effects, as 15-PGDH is upregulated in a range of tissues with aging.

### REFERENCES

1. D. S. Kehler, Age-related disease burden as a measure of population ageing. Lancet Public Health 4, e123-e124 (2019).
2. A. Y. Chang, V. F. Skirbekk, S. Tyrovolas, N. J. Kassebaum, J. L. Dieleman, Measuring population ageing: an analysis of the Global Burden of Disease Study 2017. Lancet Public Health 4, e159-e167 (2019).
3. A. T. V. Ho et al., Prostaglandin E2 is essential for efficacious skeletal muscle stem-cell function, augmenting regeneration and strength. Proc Natl Acad Sci USA 114, 6675-6684 (2017).
4. H. Chen et al., Prostaglandin E2 mediates sensory nerve regulation of bone homeostasis. Nat Commun 10, 181 (2019).
5. T. E. North et al., Prostaglandin E2 regulates vertebrate haematopoietic stem cell homeostasis. Nature 447, 1007-1011 (2007).
6. Y. Zhang et al., Inhibition of the prostaglandin-degrading enzyme 15-PGDH potentiates tissue regeneration. Science 348, aaa2340 (2015).

### Materials and Methods

### Mice

All experiments and protocols were performed in compliance with the institutional guidelines of Stanford University and Administrative Panel on Laboratory Animal Care (APLAC). Aged (>24 mo.) mice C57BL/6 were obtained from the US National Institute on Aging (NIA) for aged muscle studies, and young (2-4 mo.) wild-type C57BL/6 mice from Jackson Laboratory.

### 15-PGDH Kinetic Assay

15-PGDH activity was analyzed in tissue lysates using the BioVision PicoProbe 15-PGDH Activity Assay Kit (Cat #K562) according to the protocol of the manufacturer. Briefly, tissues were isolated and snap frozen in liquid nitrogen. Total lysates were prepared using lysis buffer (50 mM Tris-HCl pH 7.5, 150 mM NaCl, 4 mM CaCl, 1.5% Triton X-100, protease inhibitors and micrococcal nuclease) and homogenized using a FastPrep 24 homogenizer (MP Biomedicals) for 40 seconds at a speed of 6 m/s.

### Example 2. Inhibition of 15-PGDH in Aged Mice Rejuvenates Splenic Morphology

The spleen is a secondary lymphoid organ consisting of two compartments—the blood-containing red pulp region where pathogens and aged erythrocytes are removed by macrophages and the white pulp region comprising of B and T cells responsible for the adaptive immune response. The white pulp region is surrounded by a marginal zone which is involved in innate and adaptive immunity. It is comprised of stromal cells associated with a subset of macrophages and B cells that enable the capture of blood borne antigens.

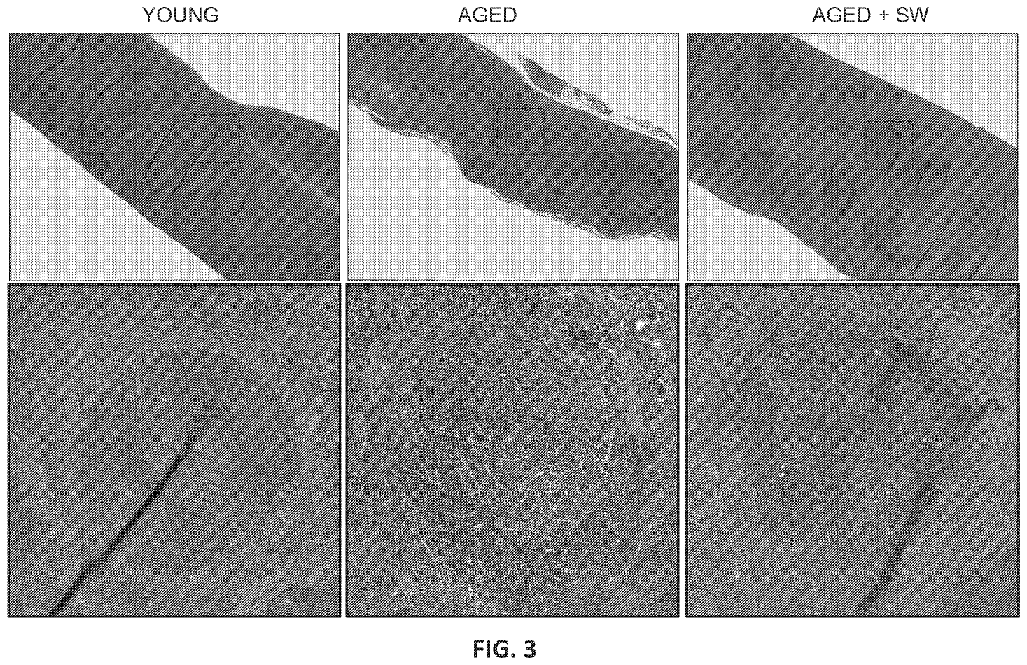

As depicted in FIG. 3, histological evaluation of aged spleens revealed an abnormal morphology with aberrant lymphoid follicular structure when compared to young mice. The aged spleens exhibited a loss of the marginal zone which separates the germinal center from the red pulp area of the spleen. Additionally, aged splenic follicles exhibited reduced cell density with areas of low cell-cell contact. Treatment of aged mice with the 15-PGDH inhibitor, SW033291, for 4 weeks rejuvenated the splenic morphology. Follicular structure was restored and the marginal zone was reestablished with increased cell density within the follicles. Since the spleen is necessary for maturation of lymphoid cell types involved in the adaptive immune response and a site of antibody selection, this data indicates the potential to use the 15-PGDH inhibitor, SW033291, to improve the immune response, decrease severity of infection in elderly, and reduce production of auto-reactive antibodies.

### Example 3. Inhibition of 15-PGDH Modulates Serum Cytokine Levels in Aged Mice

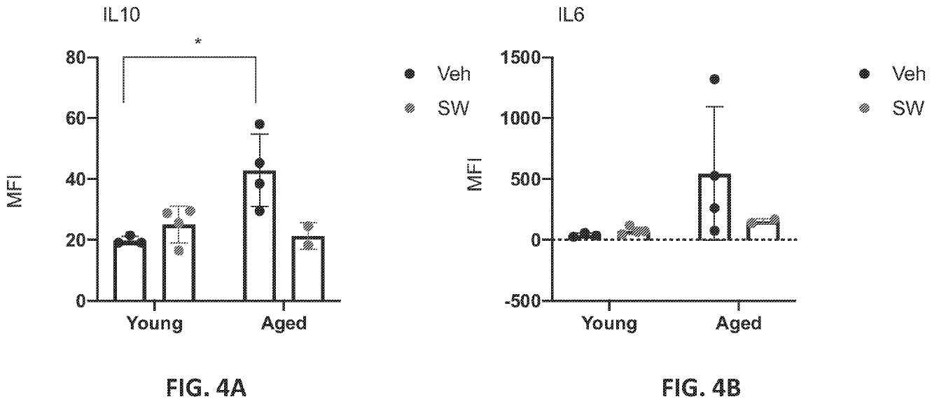

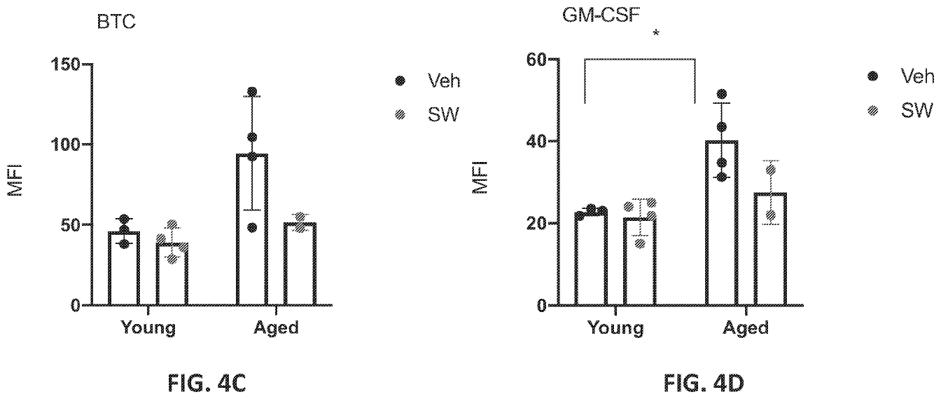

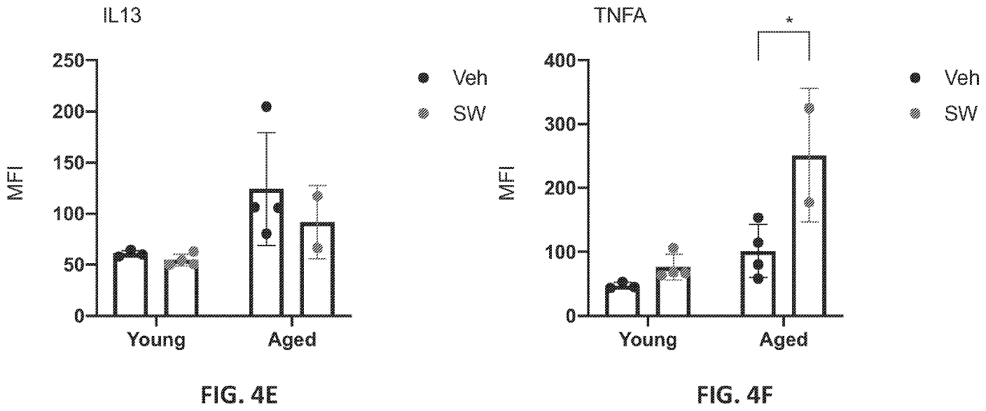

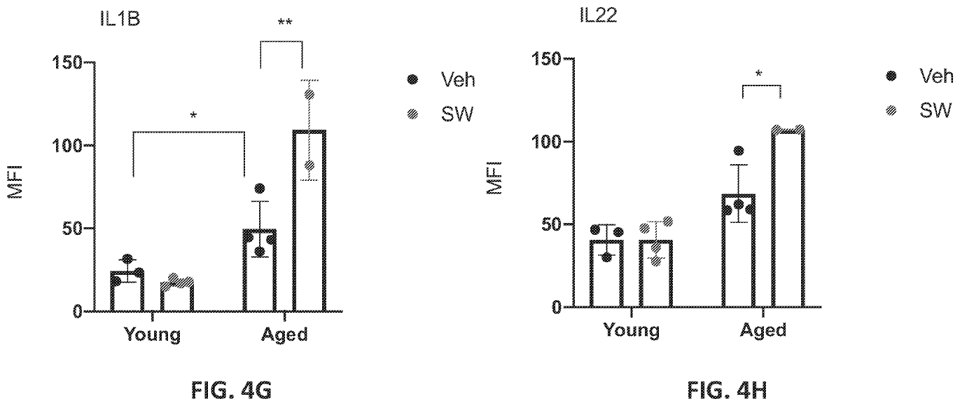

Aging is associated with an altered state of inflammation and is associated with changes in circulating levels of various cytokines. Altered cytokine signaling in aging can initiate a cascade of dysregulated signaling pathways that negatively impact the function of various organs. Age-dysregulated cytokine signaling has been implicated in increased mortality, frailty, cellular senescence, vascular aging, cognitive decline, atherosclerosis, risk of osteoarthritis, and loss of bone mineral density in aging. Using a Luminex assay of mouse sera, it was observed that aged mice exhibited an increase in circulating levels of interleukin-10 (IL10) (FIG. 4A), interleukin-6 (IL6) (FIG. 4B), betacellulin (BTC) (FIG. 4C), granulocyte-macrophage colony-stimulating factor (GM-CSF) (FIG. 4D), and interleukin-13 (IL13) (FIG. 4E). After 4-weeks of intraperitoneal administration of the 15-PGDH inhibitor, SW033291, the serum levels of all aforementioned cytokines were restored to levels similar to those of young mice, suggesting a systemic rejuvenation effect on serum cytokine levels. Furthermore, a reduction of IL6 is associated with reduced adipose tissue inflammation and reduced cellular senescence. Tumor necrosis factor-alpha (TNFA) (FIG. 4F), interleukin-1 beta (IL1B) (FIG. 4G), and interleukin-22 (IL22) (FIG. 4H) showed an increase with aging that was further increased upon treatment with the 15-PGDH inhibitor, SW033291. Increased IL22 levels are anti-inflammatory in colonic tissue and are beneficial for inflammatory bowel disease and colitis. This data suggests the use of 15-PGDH inhibition for modulating serum cytokine levels to counter dysregulated tissue signaling in aging. This systemic effect in turn ameliorates the function of other tissues.

### Example 4. Rejuvenating Aged Spleen by Administration of a 15-PGDH Inhibitor

In this example, an aged individual (e.g., a human, e.g., over 30 years of age) presents at a healthcare facility with decreased splenic function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., directly to the spleen, e.g., by injection) of the 15-PGDH inhibitor. After administration of the 15-PGDH inhibitor, the individual exhibits rejuvenation of one or more functions of the aged spleen (e.g., relative to prior to the administration, e.g., the function is rejuvenated to a level substantially similar to a level found in young spleen). The individual exhibits increased clearance of pathogens, microorganisms, cellular debris, and/or aged erythrocytes (e.g., relative to prior to the administration). The individual exhibits increased lymphoid maturation (e.g., relative to prior to the administration). The individual exhibits increased antibody generation (e.g., relative to prior to the administration).

### Example 5. Rejuvenating Aged Epithelial Tissue by Administration of a 15-PGDH Inhibitor

In this example, an aged individual (e.g., a human, e.g., over 30 years of age) presents at a healthcare facility with decreased epithelial tissue function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., directly to the epithelial tissue, e.g., by topical or subcutaneous administration) of the 15-PGDH inhibitor. After administration of the 15-PGDH inhibitor, the individual exhibits rejuvenation of one or more functions of the aged epithelial tissue (e.g., relative to prior to the administration, e.g., the function is rejuvenated to a level substantially similar to a level found in young epithelial tissue) as determined by one or more of the following measurements: transepidermal water loss, epithelial tissue hydration (e.g., skin hydration), epithelial tissue dryness (e.g., skin dryness), epithelial tissue elasticity (e.g., skin elasticity), corneocyte adhesion, ceramide concentration, pruritus, water-holding capacity, epithelial smoothness and roughness (e.g., skin smoothness and roughness), epithelial tissue wrinkles (e.g., skin wrinkles), epithelial tissue scaling (e.g., skin scaling), epithelial tissue tightness or softness (skin tightness or softness), epithelial tissue reddening and erythema formation (e.g., skin reddening and erythema formation), capillary blood flow, protection of the epithelial tissue against oxidative (including UV-induced or non-UV-induced) damage to nucleic acid, lipid, or protein, depletion of Langerhans cells after UV exposure, delayed-type hypersensitivity immune response to recall antigens in epithelial tissue such as skin, or epithelial tissue or skin sebum. The individual exhibits increased functioning of epithelial tissue including improved secretion, improved selective absorption, improved protection of underlying tissues (e.g., from radiation, desiccation, toxins, invasion by pathogens, physical trauma), improved transcellular transport, and/or improved sensing. The epithelial tissue, after 15-PGDH inhibitor treatment, exhibits levels of function that are substantially similar to levels found in young epithelial tissue. In some embodiments, after administration of the 15-PGDH inhibitor, the aged epithelial tissue exhibits enhanced skin texture and appearance, increased barrier function, increased hair growth, increased elasticity of skin, and increased stimulation of hair follicle stem cells.

### Example 6. Rejuvenating Aged Vascular Tissue by Administration of a 15-PGDH Inhibitor

In this example, an aged individual (e.g., a human, e.g., over 30 years of age) presents at a healthcare facility with decreased vascular tissue function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intravenously, intramuscularly) of the 15-PGDH inhibitor. After administration of the 15-PGDH inhibitor, the individual exhibits rejuvenation of one or more functions of the aged vascular tissue (e.g., relative to prior to the administration, e.g., the function is rejuvenated to a level substantially similar to a level found in young vascular tissue) as determined by: electrocardiography, ultrasound imaging, X-ray computed tomography and positron emission tomography, magnetic resonance imaging, angiography, contrast enhanced ultrasound, optical coherence tomography, flow-sensitive 4D-magnetic resonance imaging, bright field microscopy, fluorescence microscopy, mathematical modeling and abdominal aortic aneurysms, tissue material properties and tensile testing, tissue elasticity imaging, atomic force microscopy, flow cytometry, microfluidics, micropipette aspiration, optical microscopy, optical tweezers, or electron microscopy. The individual exhibits increased functioning of vascular tissue. The vascular tissue, after 15-PGDH inhibitor treatment, exhibits levels of function that are substantially similar to levels found in young vascular tissue.

### Example 7. Rejuvenating Aged Liver by Administration of a 15-PGDH Inhibitor

In this example, an aged individual (e.g., a human, e.g., over 30 years of age) presents at a healthcare facility with decreased liver function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., directly to the liver, e.g., by injection) of the 15-PGDH inhibitor. After administration of the 15-PGDH inhibitor, the individual exhibits rejuvenation of one or more functions of the aged liver (e.g., relative to prior to the administration, e.g., the function is rejuvenated to a level substantially similar to a level found in young liver) (e.g., as measured by administering an exogenous substance to the individual and measuring metabolite formation and/or renal excretion).

### Example 8. Rejuvenating Aged Hair by Administration of a 15-PGDH Inhibitor

In this example, an aged individual (e.g., a human, e.g., over 30 years of age) presents at a healthcare facility with hair displaying properties of aging. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., topically to the hair or scalp) of the 15-PGDH inhibitor. After administration of the 15-PGDH inhibitor, the individual exhibits rejuvenation of one or more properties of the aged hair (e.g., relative to prior to the administration, e.g., the property is rejuvenated to a level substantially similar to a level found in young hair) including hair thickness, hair quantity, and/or pigmentation.

### Example 9. Rejuvenating Aged Dental Tissue by Administration of a 15-PGDH Inhibitor

In this example, an aged individual (e.g., a human, e.g., over 30 years of age) presents at a healthcare facility with aged dental tissue. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., topical administration to the oral cavity or buccal administration) of the 15-PGDH inhibitor. After administration of the 15-PGDH inhibitor, the individual exhibits rejuvenation of one or more properties of the aged dental tissue (e.g., relative to prior to the administration, e.g., the property is rejuvenated to a level substantially similar to a level found in young dental tissue) (e.g., as measured by radiography of the dental tissue).

### Example 10. Rejuvenating Geed Small Intestine by Administration of a 15-PGDH Inhibitor

In this example, an aged individual (e.g. a human, e.g., over 30 years of age) presents at a healthcare facility with decreased small intestine function. The individual is treated with a therapeutically effective amount of a 15-PGDH inhibitor (e.g., in an amount effective to reduce 15-PGDH activity in the individual). The individual is treated by systemic administration (e.g., orally) of the 15-PGDH inhibitory, or by local administration (e.g., directly to the small intestine, e.g., by injection) of the 15-PGDH inhibitor. After administration of the 15-PGDH inhibitor, the individual exhibits rejuvenation of one or more functions of the aged small intestine (e.g., relative to prior to the administration, e.g., the function is rejuvenated to a level substantially similar to a level found in young small intestine) (e.g., as measured by a lactose tolerance test, breath tests, medical imaging, or levels of certain nutrients in a sample taken from the individual).

### Example 11. Rejuvenating Aged Colon by Administration of a 15-PGDH Inhibitor

In this example, an aged individual (e.g. a human, e.g., over 30 years of age) presents at a healthcare facility with decreased colon function. The individual is treated with a therapeutically effective amount of a 15-PGDH inhibitor (e.g., in an amount effective to reduce 15-PGDH activity in the individual). The individual is treated by systemic administration (e.g., orally) of the 15-PGDH inhibitory, or by local administration (e.g., directly to the colon, e.g., by injection) of the 15-PGDH inhibitor. After administration of the 15-PGDH inhibitor, the individual exhibits rejuvenation of one or more functions of the aged colon (e.g., relative to prior to the administration, e.g., the function is rejuvenated to a level substantially similar to a level found in young large intestine) (e.g., as measured by a physician during a general physical exam, a digital rectal exam, blood tests, x-ray, colonic transit study, colonoscopy, sigmoidoscopy, or the like).

### Example 12. Rejuvenating Aged Ovaries by Administration of a 15-PGDH Inhibitor

In this example, an aged individual (e.g., a human, e.g., over 30 years of age) presents at a healthcare facility with decreased ovarian function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., directly to the uterus, e.g., by injection) of the 15-PGDH inhibitor. After administration of the 15-PGDH inhibitor, the individual exhibits rejuvenation of one or more function of the aged ovaries or other reproductive tissues (e.g., relative to prior to the administration, e.g., the function is rejuvenated to a level substantially similar to a level found in young ovaries or other reproductive tissues) (e.g., as measured by blood or urine tests for hormone levels or egg reserves or imaging tests and procedures such as ultrasound exam, sonohysterography, hysterosalpingoraphy, hysteroscopy, or laparoscopy). The individual exhibits reduced incidence of pregnancy failure and/or chromosomally aberrant conceptions (e.g., as measured by amniocentesis or chorionic villus sampling).

### Example 13. Rejuvenating Aped Brain by Administration of a 15-PGDH Inhibitor

In this example, an aged individual (e.g., a human, e.g., over 30 years of age) presents at a healthcare facility with decreased brain function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by local administration (e.g., directly to the brain, e.g., by intracerebroventricular injection) of the 15-PGDH inhibitor. After administration of the 15-PGDH inhibitor, the individual exhibits rejuvenation of one or more functions of the aged brain (e.g., relative to prior to the administration, e.g., the function is rejuvenated to a level substantially similar to a level found in young brain) (e.g., as measured by magnetic resonance imagining (MRI) and positron emission tomography (PET) scan). The individual exhibits increased brain size (e.g., relative to prior to the administration). The individual exhibits increased level of neurotransmitters or hormones (e.g., relative to prior to the administration) (e.g., as measured by microdialysis technique coupled to high performance liquid chromatography (HPLC) or by electrochemical, optical or magnetic methods). The individual exhibits improved cognitive performance (e.g., relative to prior to the administration) (e.g., as measured by Morris water maze test).

### Example 14. Rejuvenating Aged Skin by Administration of a 15-PGDH Inhibitor

In this example, an aged individual (e.g., a human, e.g., over 30 years of age) presents at a healthcare facility with decreased epidermal tissue function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by local administration (e.g., directly to the epidermal tissue, e.g., by topical administration or intradermal injection) of the 15-PGDH inhibitor. After administration of the 15-PGDH inhibitor, the individual exhibits rejuvenation of one or more functions of the aged epidermal tissue (e.g., relative to prior to the administration, e.g., the function is rejuvenated to a level substantially similar to a level found in young epidermal tissue) (e.g., as measured by spectroscopic and imaging methodologies). The skin of the individual exhibits increased strength and elasticity (e.g., relative to prior to the administration) (e.g., as measured by applying skin deformation either in the plane of the skin e.g., torsion, or horizontally to it e.g., suction or indentation). The skin of the individual exhibits increased hair follicle stem cells (e.g., relative to prior to the administration).

### Example 15. Rejuvenating Aged Cardiac Muscle by Administration of a 15-PGDH Inhibitor

In this example, an aged individual (e.g., a human, e.g., over 30 years of age) presents at a healthcare facility with decreased cardiac muscle function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intramuscularly) of the 15-PGDH inhibitor. After administration of the 15-PGDH inhibitor, the individual exhibits rejuvenation of one or more functions of the aged cardiac muscle (e.g., relative to prior to the administration. e.g., the function is rejuvenated to a level substantially similar to a level found in young cardiac muscle) as determined by: echocardiogram, transesophageal echocardiography (TEE), electrocardiogram (ECG or EKG), magnetic resonance imaging (MRI), CT scan, exercise cardiac stress test, pharmacologic stress test, tilt test, ambulatory rhythm monitoring tests, or coronary angiogram. The cardiac muscle, after 15-PGDH inhibitor treatment, exhibits levels of function such as pumping oxygenated blood to the other body parts; pumping hormones and other vital substances to different parts of the body; receiving deoxygenated blood and carrying metabolic waste products from the body and pumping it to the lungs for oxygenation; or maintaining blood pressure that are substantially similar to levels of function found in young cardiac muscle. The aged individual, after administration of the 15-PGDH inhibitor, exhibits decreased fibrosis or a exhibits a fibrosis level that is substantially similar to a fibrosis level in young cardiac muscle.

### Example 16. Rejuvenating Aged Bone by Administration of a 15-PGDH Inhibitor

In this example, an aged individual (e.g., a human, e.g., over 30 years of age) presents at a healthcare facility with decreased bone function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intraosseous infusion) of the 15-PGDH inhibitor. After administration of the 15-PGDH inhibitor, the individual exhibits rejuvenation of one or more functions of the aged bone (e.g., relative to prior to the administration, e.g., the function is rejuvenated to a level substantially similar to a level found in young bone as determined by: mechanical methods (e.g., whole-bone mechanical testing, bulk tissue specimen mechanical testing, microbeam mechanical testing, microindentation, or nanoindentation); imaging methods (e.g., CT, MRI, NMR, FTR, Raman imaging, or scanning electron microscopy); chemical or physical methods (gravimetric analysis or chemical analysis of collagen crosslinks); bone densitometry. The aged bone, after 15-PGDH inhibitor treatment, exhibits levels of function including mechanical support and movement; hematopoiesis; storage of mineral or fat; stabilizing pH or calcium; hormone secretion; lubrication; or damages repair that are substantially similar to levels of function found in young bone. In some cases, the aged bone, after administration of the 15-PGDH inhibitor, exhibits decreased fibrosis or exhibits a fibrosis level that is substantially similar to a fibrosis level in young bone. In some cases, the aged individual, after administration of the 15-PGDH inhibitor, exhibits increased growth of bone.

### Example 17. Rejuvenating Aged Sensory Organs by Administration of a 15-PGDH Inhibitor

In this example, an aged individual (e.g., a human, e.g., over 30 years of age) presents at a healthcare facility with decreased sensory organ (e.g., eye, ear, nose, tongue) function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intraocularly or intranasally) of the 15-PGDH inhibitor. After administration of the 15-PGDH inhibitor, the individual exhibits rejuvenation of one or more functions of the aged sensory organ (e.g., relative to prior to the administration, e.g., the function is rejuvenated to a level substantially similar to a level found in young sensory organ as determined by: visual test, hearing test, olfactory test, physical fitness or balance test, or any other tests that examine internal or external senses or stimuli. The sensory organ, after 15-PGDH inhibitor treatment, exhibits levels of function including sensing stimuli such as physical stimuli such as pressure and vibration, sensation of sound, or body position (balance); light (visible electromagnetic radiation); chemical stimuli such as taste or smell; pain: temperature; or other internal stimuli that are substantially similar to levels of function found in young sensory organ. In some cases, the aged individual, after administration of the 15-PGDH inhibitor, exhibits decreased level of dry eye disease, lacrimal gland inflammation, or macular degeneration.

### Example 18. Rejuvenating Aged Kidney by Administration of a 15-PGDH Inhibitor

In this example, an aged individual (e.g., a human, e.g., over 30 years of age) presents at a healthcare facility with decreased kidney function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intraosseous infusion) of the 15-PGDH inhibitor. After administration of the 15-PGDH inhibitor, the individual exhibits rejuvenation of one or more functions of the aged kidney (e.g., relative to prior to the administration, e.g., the function is rejuvenated to a level substantially similar to a level found in young kidney as determined by: clinical assessment; urine tests; blood tests (e.g., glomerular filtration rate); medical imaging (e.g., CT scan); or biopsy. The kidney, after 15-PGDH inhibitor treatment, exhibits levels of function including formation of urine (e.g., filtration, reabsorption, secretion, or excretion); hormone secretion; blood pressure regulation; acid-base balance; or regulation of osmolality that are substantially similar to levels of function found in young kidney. In certain instances, the aged individual, after administration of the 15-PGDH inhibitor, exhibits decreased level of kidney disease such as chronic kidney disease, nephritic and nephrotic syndromes, acute kidney injury, pyelonephritis, or kidney cancer.

### Example 19. Rejuvenating Aged Thyroid by Administration of a 15-PGDH Inhibitor

In this example, an aged individual (e.g., a human, e.g., over 30 years of age) presents at a healthcare facility with decreased thyroid function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intravenously) of the 15-PGDH inhibitor. After administration of the 15-PGDH inhibitor, the individual exhibits rejuvenation of one or more functions of the aged thyroid (e.g., relative to prior to the administration, e.g., the function is rejuvenated to a level substantially similar to a level found in young thyroid as determined by: blood tests for measuring thyroid-stimulating hormone (TSH), triiodothyronine (T3), thyroxine (T4), or calcitonin; antibody tests for detecting thyroid hormones; or radioactive iodine uptake. The thyroid, after 15-PGDH inhibitor treatment, exhibits levels of function including regulating, producing, and secreting hormones that are substantially similar to levels of function found in young thyroid. In certain instances, the aged individual, after administration of the 15-PGDH inhibitor, exhibits decreased level of thyroid disease such as hyperthyroidism, hypothyroidism, Hashimoto's thyroiditis, Graves' disease, goiter, thyroid nodule, or thyroid cancer.

### Example 20. Rejuvenating Aged Lung by Administration of a 15-PGDH Inhibitor

In this example, an aged individual (e.g., a human, e.g., over 30 years of age) presents at a healthcare facility with decreased lung function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., inhalation or intranasally) of the 15-PGDH inhibitor. After administration of the 15-PGDH inhibitor, the individual exhibits rejuvenation of one or more functions of the aged lung (e.g., relative to prior to the administration, e.g., the function is rejuvenated to a level substantially similar to a level found in young lung as determined by: evaluating lung capacity for volume or air inhaled or exhaled; pulmonary plethysmographs; spirometry; lung diffusing capacity; pulse oximetry; lung imaging; bronchoscopy; or thoracotomy. The lung, after 15-PGDH inhibitor treatment, exhibits levels of function including gas exchange between lung and blood; protection against respiratory pathogen or infection; maintaining homeostasis of pressure or acid-base in blood; or speech by providing air and airflow for the creation of vocal sound that are substantially similar to levels of function found in young lung. In certain instances, the aged individual, after administration of the 15-PGDH inhibitor, exhibits decreased level of lung disease such as inflammation, infection, blood-supply change, obstructive lung disease, restrictive lung disease, congenital disorder, pneumothorax, lung nodule, or lung cancer. In certain instances, the aged individual, after administration of the 15-PGDH inhibitor, exhibits decreased level of fibrosis in the aged lung.

### Example 21. Rejuvenating Aged Smooth Muscle by Administration of a 15-PGDH Inhibitor

In this example, an aged individual (e.g., a human, e.g., over 30 years of age) presents at a healthcare facility with decreased smooth muscle function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intramuscularly) of the 15-PGDH inhibitor. After administration of the 15-PGDH inhibitor, the individual exhibits rejuvenation of one or more functions of the aged smooth muscle (e.g., relative to prior to the administration, e.g., the function is rejuvenated to a level substantially similar to a level found in young smooth muscle as determined by: blood test for measuring antibody associated with smooth muscle or medical imaging. The smooth muscle, after 15-PGDH inhibitor treatment, exhibits levels of function including contraction and relaxation, which lead to movement of the digestive tract, movement of the autonomous nervous system (e.g., for breathing), or regulating homeostasis such as raising skin hair follicles for regulating body temperature that are substantially similar to levels of function found in young smooth muscle. In certain instances, the aged individual, after administration of the 15-PGDH inhibitor, exhibits decreased level of smooth muscle disease such as multisystemic smooth muscle dysfunction syndrome, blood vessel disorders, atherosclerosis, or inflammation. In certain instances, the aged individual, after administration of the 15-PGDH inhibitor, exhibits decreased level of fibrosis in the aged smooth muscle.

### Example 22. Rejuvenating Aged Blood by Administration of a 15-PGDH Inhibitor

In this example, an aged individual (e.g., a human, e.g., over 30 years of age) presents at a healthcare facility with decreased blood function. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally or intravenously) of the 15-PGDH inhibitor, or by local administration (e.g., intravenously or intraventricularly) of the 15-PGDH inhibitor. After administration of the 15-PGDH inhibitor, the individual exhibits rejuvenation of one or more functions of the aged blood (e.g., relative to prior to the administration, e.g., the function is rejuvenated to a level substantially similar to a level found in young blood as determined by: metabolic panel for measuring metabolites such as electrolytes, calcium, glucose, sodium, potassium, carbon dioxide, chloride, blood urea nitrogen (BUN), creatinine, albumin, total protein, alkaline phosphatase, alkaline aminotransferase, aspartate aminotransferase, or bilirubin; lipid panel; thyroid panel; enzyme markers; coagulation panel; dehydroepiandrosterone (DHEA)-sulfate serum test; C-reactive protein test; or circulating cytokines (e.g., by detecting and measuring serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1 b), interleukin-22 (IL22), and any combination thereof.). The blood, after 15-PGDH inhibitor treatment, exhibits levels of function including supply of oxygen to tissues (bound to hemoglobin in red blood cells); supply of nutrients such as glucose, amino acids, or fatty acids (dissolved in the blood or bound to plasma proteins); removal of waste such as carbon dioxide, urea, or lactic acid; immune response, including circulation of white blood cells and detection of foreign material by antibodies; coagulation, messenger function, including transport of hormones; or regulation of core body temperature that are substantially similar to levels of function found in young blood. In certain instances, the aged individual, after administration of the 15-PGDH inhibitor, exhibits decreased level of blood disease such as anemia, hemophilia, blood clots, and blood cancers such as leukemia, lymphoma, or myeloma. In some embodiments, after administration of the 15-PGDH inhibitor, the aged blood exhibits restored or rejuvenated serum cytokine levels that are substantially similar to serum cytokine levels found in a young individual.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of rejuvenating a function of an aged non-skeletal muscle tissue or aged non-skeletal muscle organ in an individual, the method comprising: administering to the individual or to the aged non-skeletal muscle tissue or aged non-skeletal muscle organ a 15-hydroxyprostaglandin dehydrogenase (15-PGDH) inhibitor in an amount effective to inhibit 15-PGDH activity and/or reduce 15-PGDH levels in the individual, thereby rejuvenating the function of the aged non-skeletal muscle tissue or the aged non-skeletal muscle organ in the individual.

2. The method of claim 1, wherein, after the administering, the function is rejuvenated relative to a function of the aged non-skeletal muscle tissue or aged non-skeletal muscle organ prior to administration of the 15-PGDH inhibitor.

3. The method of claim 1 or 2, wherein, after the administering, the function is rejuvenated by at least about 10% relative to a function of the aged non-skeletal muscle tissue or aged non-skeletal muscle organ prior to administering the 15-PGDH inhibitor.

4. The method of any one of claims 1-3, wherein, after the administering, the function of the aged non-skeletal muscle tissue or the aged non-skeletal muscle organ is rejuvenated to a level substantially similar to a level of a function of a young non-skeletal muscle tissue or a young non-skeletal muscle organ.

5. The method of any one of claims 1-4, wherein, after the administering, the function of the aged non-skeletal muscle tissue or the aged non-skeletal muscle organ is rejuvenated to a level within at least about 50% of a level of a function of a young non-skeletal muscle tissue or a young non-skeletal muscle organ.

6. The method of any one of claims 1-5, wherein, after the administering, a level of prostaglandin E2 (PGE2) in the aged non-skeletal muscle tissue or aged non-skeletal muscle organ is increased relative to a level of PGE2 in the aged non-skeletal muscle tissue or aged non-skeletal muscle organ prior to the administering.

7. The method of any one of claims 1-6, wherein, after the administering, a level of PGE2 in the aged non-skeletal muscle tissue or aged non-skeletal muscle organ is increased by at least about 10% relative to a level of PGE2 in the aged non-skeletal muscle tissue or aged non-skeletal muscle organ prior to the administering.

8. The method of any one of claims 1-7, wherein, after the administering, a level of PGE2 in the aged non-skeletal muscle tissue or aged non-skeletal muscle organ is increased to a level substantially similar to a level of PGE2 present in a young non-skeletal muscle tissue or a young non-skeletal muscle organ.

9. The method of any one of claims 1-8, wherein, after the administering, a level of PGE2 in the aged non-skeletal muscle tissue or aged non-skeletal muscle organ is increased to a level within at least about 50% of a level of PGE2 present in a young non-skeletal muscle tissue or a young non-skeletal muscle organ.

10. The method of any one of claims 1-9, wherein the administering increases systemic levels of PGE2 in the individual.

11. The method of any one of claims 1-10, wherein the administering results in a rejuvenation of serum cytokines to levels substantially similar to serum cytokine levels found in a young individual.

12. The method of claim 11, wherein the serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-a), interleukin-1 beta (IL1 b), interleukin-22 (IL22), and any combination thereof.

13. The method of any one of claims 1-12, wherein the aged non-skeletal muscle tissue or the aged non-skeletal muscle organ is selected from the group consisting of: epidermal tissue, epithelial tissue, vascular tissue, cardiac muscle, brain, bone, cartilage, sensory organs (e.g., organs involved in sight, hearing, taste, smell, or touch), kidney, thyroid, lung, smooth muscle, brown fat, spleen, liver, heart, small intestine, colon, skin, ovaries and other reproductive tissues, hair, dental tissue, blood, cochlea, and any combination thereof.

14. The method of claim 13, wherein the aged non-skeletal muscle tissue or the aged non-skeletal muscle organ is spleen.

15. The method of claim 14, wherein, after the administering, the spleen exhibits improved clearance of pathogens, microorganisms, cellular debris, and/or aged erythrocytes from the blood, improved or enhanced maturation of lymphoid cell types, increased antibody generation, or any combination thereof.

16. The method of any one of claims 1-15, wherein the administering results in: increased adaptive and/or innate immune response in the individual relative to prior to the administering, decreased severity of infection in the individual relative to prior to the administering, decreased production of auto-antibodies relative to prior to the administering, treatment of or improvement of symptoms associated with type 11 diabetes, treatment of or improvement of symptoms associated with rheumatoid arthritis, or any combination thereof.

17. The method of claim 13, wherein the aged non-skeletal muscle tissue or the aged non-skeletal muscle organ is skin.

18. The method of claim 17, wherein, after the administering, the skin exhibits enhanced skin condition, exhibits increased barrier function, supports increased hair growth, counters baldness, exhibits increased stimulation of hair follicle stem cells, exhibits increased elasticity of skin, treatment or improvement of symptoms or effects associated with alopecia, treatment or improvement of symptoms or effects associated with pattern baldness, or any combination thereof.

19. The method of claim 13, wherein the aged non-skeletal muscle tissue or the aged non-skeletal muscle organ is brain.

20. The method of claim 19, wherein, after the administering, the brain exhibits increased brain size, exhibits increased grey matter, exhibits increased amount of neuronal cells, exhibits increased neuronal volume, exhibits improved cognitive performance, exhibits improved memory performance, exhibits increased level of neurotransmitters such as dopamine, serotonin and other brain-derived neurotrophic factors, exhibits increased level of hormones, exhibits reduced risk of stroke, white matter lesions, or dementia, exhibits reduced risk of Alzheimer's or Parkinson's disease, or any combination thereof.

21. The method of claim 13, wherein the aged non-skeletal muscle tissue or the aged non-skeletal muscle organ is bone.

22. The method of claim 21, wherein, after the administering, the bone exhibits improved mechanical support and movement, exhibits improved angiogenesis, exhibits improved storage of mineral or fat, exhibits improved stabilization of pH or calcium, exhibits improved hormone secretion, exhibits improved lubrication, exhibits decreased fibrosis, or any combination thereof.

23. The method of claim 13, wherein the aged non-skeletal muscle tissue or the aged non-skeletal muscle organ is kidney.

24. The method of claim 23, wherein, after the administering, the kidney is protected from ischemic renal injury, exhibits increased vasodilation, exhibits increased renal blood flow, exhibits reduced biomarkers of renal injury, exhibits improved formation of urine, exhibits improved filtration, exhibits improved reabsorption, exhibits improved secretion, exhibits improved excretion, exhibits improved hormone secretion, exhibits improved blood pressure regulation, exhibits improved acid-base balance, exhibits improved regulation of osmolality, exhibits decreased levels of kidney disease, or any combination thereof.

25. The method of claim 13, wherein the aged non-skeletal muscle tissue or the aged non-skeletal muscle organ is thyroid.

26. The method of claim 25, wherein, after the administering, the thyroid exhibits improved regulation, production, and/or secretion of hormones and/or exhibits decreased levels of thyroid disease.

27. The method of claim 13, wherein the aged non-skeletal muscle tissue or the aged non-skeletal muscle organ is lung.

28. The method of claim 27, wherein, after the administering, the lung exhibits decreased levels of lung disease, exhibits decreased levels of fibrosis, exhibits increased lung capacity, or any combination thereof.

29. The method of claim 13, wherein the aged non-skeletal muscle tissue or the aged non-skeletal muscle organ is cardiac muscle and/or heart.

30. The method of claim 29, wherein, after the administering, the cardiac muscle and/or heart exhibits increased or enhanced cardiac muscle tissue functions, improved pumping of oxygenated blood to other body parts, improved pumping of hormones and other vital substances to different parts of the body, improvement in receiving deoxygenated blood and carrying metabolic waste products from the body and pumping it to the lungs for oxygenation, improved maintenance of blood pressure, exhibits decreased fibrosis, or any combination thereof.

31. The method of claim 13, wherein the aged non-skeletal muscle tissue or the aged non-skeletal muscle organ is smooth muscle.

32. The method of claim 31, wherein, after the administering, the smooth muscle exhibits decreased levels of smooth muscle disease, exhibits decreased levels of fibrosis, exhibits increased angiogenesis or vasculogenesis, and/or is more sensitive to temperature changes and/or adrenalin level changes.

33. The method of claim 13, wherein the aged non-skeletal muscle tissue or the aged non-skeletal muscle organ is liver.

34. The method of claim 33, wherein, after the administering, the liver exhibits improved maintenance of whole-body homeostasis through regulation of metabolism, xenobiotic, and endobiotic clearance and molecular biosynthesis, exhibits improved formation and excretion of bile, exhibits improved regulation of carbohydrate homeostasis, lipid synthesis and secretion of plasma lipid proteins, exhibits improved control of cholesterol metabolism, exhibits improved formation of urea, serum albumin, clotting factors, enzymes, and other proteins, exhibits decreased fibrosis, exhibits decreased fatty acid storage, exhibits decreased liver adipose content, or any combination thereof.

35. The method of claim 13, wherein the aged non-skeletal muscle tissue or the aged non-skeletal muscle organ is small intestine.

36. The method of claim 35, wherein, after the administering, the small intestine exhibits increased production of lactase, exhibits reduced growth of certain bacteria, improved digestion of dairy products, improved absorption of nutrients, or any combination thereof.

37. The method of claim 13, wherein the aged non-skeletal muscle tissue or the aged non-skeletal muscle organ is colon.

38. The method of claim 37, wherein, after the administering, the colon exhibits increased or enhanced peristalsis and/or recovery from ulcerative colitis including diarrhea or gastrointestinal bleeding.

39. The method of claim 13, wherein the aged non-skeletal muscle tissue or the aged non-skeletal muscle organ is ovaries or other reproductive tissues/organs.

40. The method of claim 39, wherein, after the administering, the ovaries or other reproductive tissues/organs exhibit reduced or halted ovary decline and/or exhibits a reduction in pregnancy failure and/or number of chromosomally aberrant conceptions.

41. The method of claim 13, wherein the aged non-skeletal muscle tissue or the aged non-skeletal muscle organ is hair.

42. The method of claim 41, wherein a property of the aged hair is rejuvenated, the property selected from the group consisting of: pigmentation, diameter, curvature, stretching, bending, torsional rigidity, lipid composition, and any combination thereof.

43. The method of claim 13, wherein the aged non-skeletal muscle tissue or the aged non-skeletal muscle organ is dental tissue.

44. The method of claim 43, wherein, after the administering, the dental tissue exhibits an increased ratio of dentin to dental pulp and/or a reduced level or reversal of the conversion of dental pulp to dentin.

45. The method of claim 13, wherein the aged non-skeletal muscle tissue or the aged non-skeletal muscle organ is blood.

46. The method of claim 45, wherein, after the administering, the blood exhibits improved supply of oxygen to tissues, exhibits improved supply of nutrients to tissues, exhibits improved removal of waste, exhibits improved immune response, exhibits improved circulation of white blood cells, exhibits improved detection of foreign material by antibodies, exhibits improved coagulation, exhibits improved transport of hormones, exhibits improved regulation of core body temperature, exhibits decreased levels of blood diseases, or any combination thereof.

47. The method of claim 13, wherein the aged non-skeletal muscle tissue or the aged non-skeletal muscle organ is a sensory organ or the cochlea.

48. The method of claim 47, wherein, after the administering, the sensory organ exhibits enhanced or improved sensory function (e.g., sight, smell, taste, hearing) and/or reduction or treatment of dry eye disease, lacrimal gland inflammation, or macular degeneration.

49. The method of claim 13, wherein the aged non-skeletal muscle tissue or the aged non-skeletal muscle organ is epithelial tissue.

50. The method of claim 49, wherein, after the administering, the epithelial tissue exhibits improved secretion, exhibits improved selective absorption, exhibits improved protection of underlying tissues (e.g., from radiation, desiccation, toxins, invasion by pathogens, physical trauma), exhibits improved transcellular transport, exhibits improved sensing, or any combination thereof.

51. The method of claim 13, wherein the aged non-skeletal muscle tissue or the aged non-skeletal muscle organ is vascular tissue.

52. The method of claim 51, wherein, after the administering, the vascular tissue exhibits improved vasodilation, improved angiogenesis, improved access of nutrients to tissue, improved blood transport, or any combination thereof.

53. The method of any one of claims 1-52, wherein the individual has one or more biomarkers of aging.

54. The method of claim 53, wherein the one or more biomarkers of aging is selected from the group consisting of: an increase in 15-PGDH levels relative to a young individual, a decrease in PGE2 levels relative to a young individual, an increase in a PGE2 metabolite relative to a young individual, an increase or a greater accumulation of senescent cells relative to a young individual, an increase in expression of one or more atrogenes relative to a young individual, a decrease in mitochondria biogenesis and/or function relative to a young individual, an increase in transforming growth factor pathway signaling relative to a young individual, and any combination thereof.

55. The method of any one of claims 1-54, wherein the aged non-skeletal muscle tissue or the aged non-skeletal muscle organ has an increased accumulation of senescent cells relative to a young non-skeletal muscle tissue or a young non-skeletal muscle organ.

56. The method of claim 55, wherein the senescent cells express one or more senescent markers.

57. The method of claim 55 or 56, wherein the senescent cells have an increased level of one or more senescent markers relative to non-senescent cells.

58. The method of claim 56 or 57, wherein the one or more senescent markers is selected from the group consisting of: p15Ink4b, p16Ink4a, p19Arf, p21, Mmp13, Il1a, Il1b, and Il6.

59. The method of any one of claims 55-58, wherein the senescent cells are macrophages.

60. The method of any one of claims 1-59, wherein the 15-PGDH inhibitor is selected from the group consisting of: a small molecule compound, a blocking antibody, a nanobody, and a peptide.

61. The method of any one of claims 1-60, wherein the 15-PGDH inhibitor is SW033291.

62. The method of any one of claims 1-59, wherein the 15-PGDH inhibitor is selected from the group consisting of: an antisense oligonucleotide, microRNA, siRNA, and shRNA.

63. The method of any one of claims 1-62, wherein the individual is a human.

64. The method of any one of claims 1-63, wherein the individual is at least 30 years of age.

65. The method of any one of claims 1-64, wherein the 15-PGDH inhibitor reduces or blocks 15-PGDH expression.

66. The method of any one of claims 1-65, wherein the 15-PGDH inhibitor reduces or blocks enzymatic activity of 15-PGDH.

67. The method of any one of claims 1-66, wherein the administering results in decreased levels of a PGE2 metabolite in the aged non-skeletal muscle tissue or aged non-skeletal muscle organ relative to the aged non-skeletal muscle tissue or the aged non-skeletal muscle muscle prior to the administering of the 15-PGDH inhibitor and/or to a level that is substantially similar to a level present in young non-skeletal muscle tissue or a young non-skeletal muscle organ.

68. The method of claim 67, wherein the PGE2 metabolite is selected from the group consisting of: 15-keto PGE2 and 13,14-dihydro-15-keto PGE2.

69. The method of any one of claims 1-68, wherein the administering comprises systemic administration.

70. The method of claim 69, wherein the systemic administration is oral administration or intraperitoneal administration.

71. The method of any one of claims 1-70, wherein the administering comprises local administration.

72. The method of any one of claims 1-71, wherein the administering comprises single-dose administration.

73. The method of any one of claims 1-71, wherein the administering comprises administering the 15-PGDH inhibitor periodically.
